(19) United States
(12) Patent Application Publication
(10) Pub. No.: US 2024/0144177 A1
(43) Pub. Date: May 2, 2024

(54) SYSTEM, METHOD, PROCESS, AND MECHANISM FOR FACILITATING ORDER PLACEMENT, CONSOLIDATION, AND FULFILLMENT OF MULTI-VENDOR ORDERS VIA A DRIVE-THROUGH COMPLEX

(71) Applicant: Pavilion Capital, LLC, Charlotte, NC (US)

(72) Inventors: Richard M. Davies, Charlotte, NC (US); Daniel Montano, Charlotte, NC (US); James Farnell, Laguna Niguel, CA (US); Marnie Harris, Decatur, GA (US); Andrew McKinney, Charlotte, NC (US); Dave Meadows, Charlotte, NC (US); Jim Gamble, Charlotte, NC (US); Jeff Blake, Huntersville, NC (US)

(73) Assignee: Pavilion Capital, LLC, Charlotte, NC (US)

(21) Appl. No.: 18/281,254

(22) PCT Filed: Mar. 11, 2022
(86) PCT No.: PCT/US2022/020002

§ 371 Date: Sep. 8, 2023

Related U.S. Application Data
(60) Provisional application No. 63/160,620, filed on Mar. 12, 2021.
(60) Provisional application No. 63/214,415, filed on Jun. 24, 2021.
(60) Provisional application No. 63/239,046, filed on Aug. 31, 2021.

(51) Int. Cl. G06Q 10/087

(52) U.S. Cl. CPC G06Q 10/087

(57) ABSTRACT

A system, method, process, and mechanism for fulfilling multi-vendor orders within a drive-through complex with a vertical transport system that delivers the multi-vendor orders to pick-up stations located below the floors with the vendors. The system receives an order from a user device of a user, wherein the order includes at least two sub-orders. The sub-orders are processed based at least on the preparation times. The sub-orders are tracked and an available drive-through lane with an available pick-up station is determined for the order including the sub-orders. Notifications may be sent to a user or third party user for order pick-up. When the order is complete, it is consolidated and the order is transferred to the available pick-up station.

### CROSS REFERENCE AND PRIORITY CLAIM UNDER 35 U.S.C. § 119

This application claims priority to U.S. Provisional Application No. 63/160,620 filed on Mar. 12, 2021; U.S. Provisional Application No. 63/214,415 filed on Jun. 24, 2021; and U.S. Provisional Application No. 63/239,046 filed on Aug. 31, 2021 all of which are entitled “System, Method, Process, and Mechanism for Facilitating Order Placement, Consolidation, and Fulfillment of Multi-vendor Orders via a Drive-Through Complex,” all of which are assigned to the assignee hereof and hereby expressly incorporated by reference herein.

### FIELD

The present invention relates to a multi-vendor drive-through complex, and more particularly to, a system, method, computer program product, and mechanisms that facilitate order placement, consolidation, and fulfillment of multi-vendor orders.

### BACKGROUND

Conventional systems do not have the capability to facilitate fulfilment of multi-vendor orders in an efficient manner. As such, there exists a need for a system, a method, a computer program product, and mechanisms to facilitate order placement, consolidation, and fulfillment of multi-vendor orders.

### BRIEF SUMMARY

The following presents a summary of certain embodiments of the invention. This summary is not intended to identify key or critical elements of all embodiments nor delineate the scope of any or all embodiments. Its sole purpose is to present certain concepts and elements of one or more embodiments in a summary form as a prelude to the more detailed description that follows.

Embodiments of the present invention address the above needs and/or achieve other advantages by providing a systems, methods, computer program products, and mechanisms for fulfillment of customers' multi-vendor orders. The system embodiments may comprise one or more memory devices having computer readable program code stored thereon, a communication device, and one or more processing devices operatively coupled to the one or more memory devices, wherein the one or more processing devices are configured to execute the computer readable program code to carry out the invention. In computer program product embodiments of the invention, the computer program product comprises at least one non-transitory computer readable medium comprising computer readable instructions for carrying out the invention. Computer implemented method embodiments of the invention may comprise providing a computing system comprising a computer processing device and a non-transitory computer readable medium, where the computer readable medium comprises configured computer program instruction code, such that when said instruction code is operated by said computer processing device, said computer processing device performs certain operations to carry out the invention.

In some embodiments, the present invention is configured for receiving an order from a user device of a user, where the order comprises at least two sub-orders, consolidating and processing the order received from the user, calculating one or more preparation times associated with the order, wherein calculating the one or more preparation times comprises prioritizing the at least sub-orders, transmitting the one or more preparation times to one or more vendor systems, tracking progress of the order, assembling the order from multiple vendors, determining an available drive-through lane with available pick-up stations (e.g., kiosks, lockers, human stations, robotic handling and delivery stations, or the like), instructing delivery of the order to the pick-up stations, such as through the use of horizontal transport systems (e.g., floor transport system, such as conveyers, tracks, etc.) and vertical transport systems (e.g., elevators for humans or robots, dummy waiters, or the like), and/or robotic delivery systems (e.g., robots that move around a floor and between floors to receive orders from vendors and deliver the orders to the pick-up stations), and transmitting one or more notifications to the one or more users and/or one or more third party users.

In some embodiments, the present invention is a multi-vendor drive-through complex, where the multi-vendor drive-through complex is a building that is adapted to allow multiple vendors to store and/or prepare products (e.g., goods, services, or the like) and allow users (e.g., one or more customer users, one or more third party users, or the like) to pick-up one or more orders comprising one or more sub-orders from multiple vendors. In some embodiments, the building comprises one or more drive-through lanes with a routing mechanism, where the one or more users (e.g., customer users and/or one or more third party users, or the like) can pick-up the one or more orders from one or more pick-up stations present in the drive-through lanes. In some embodiments, one or more of the drive-through lanes may be dedicated lanes for user operated vehicles, autonomous delivery vehicles, one or more lanes or locations may be dedicated for bikes, scooters, and/or locations for walk up customers on the ground (e.g., under the first floor), and/or drone locations may be located on a roof or other upper level of the complex. In some embodiments, one or more dumbwaiters and/or robotic delivery systems may pick-up orders from multiple vendors located on one or more floors and deliver the orders to the one or more pick-up stations present in the one or more lanes or locations on the ground floor and/or to the drone locations on the upper floors.

In some embodiments, the building further comprises one or more parking spaces which allow one or more users (e.g., customer users, one or more third party users, or the like) to wait to pick-up the order (e.g., when there is an error in the order, when part of the order is not ready, or the like). Moreover, due to the traffic that may be running through the complex at times, the complex may have additional lanes and traffic control elements, which may communicate with and/or work independently from or in conjunction with traffic control elements (e.g., lights, signs, merging, or the like) in the surrounding streets (e.g., corner streets, or the like). As such, the complex may control the timing of when users (e.g., user vehicles, bicycle, scooter, lanes), autonomous delivery vehicles (e.g., wheeled, tracked, or the like), and/or drones enter and/or leave the complex, as will be discussed herein.

In some embodiments the drive-through complex includes a drive-through medical services section with medical services stations (e.g., one or more drive-in bays, or the like) that are used to provide medical services to patients. The drive-through complex may also include one or more floors located at least partially above or below the drive-through medical services section, including a medical distribution vendor (e.g., pharmacy, or the like). The one or more floors may also include medical offices and/or additional medical service stations (e.g., exam rooms, operating rooms, diagnostic rooms, or the like) that provide higher level medical services than the drive-through medical services section. A medical application may be used to receive medical information from a user (e.g., patient, caregiver, medical personal, or the like), determine a decisioning action for the patient, and route the patient to the drive-through medical service stations or the additional medical service stations located on the floors of the complex. The drive-through medical complex may further include a parking garage that has designated parking floors for patients seeking treatment from designated medical floors, and may limit access to the designated parking floors to only patients that are to receive medical services on the adjacent medical floor.

One embodiment of the present disclosure comprises a multi-vendor drive-through complex for multiple vendors providing goods to users. The multi-vendor drive-through complex comprises one or more floors occupied by one or more vendors, one or more drive-through lanes located below the one or more floors, pick-up stations in the one or more drive-through lanes for storing orders. The pick-up stations are automated pick-up stations and the orders comprise one or more goods provided by the one or more vendors. One or more vertical transport systems deliver the orders to the one or more pick-up stations from the one or more floors above the drive-through lanes.

In further accord with embodiments, at least one floor of the one or more floors is occupied by two or more vendors.

In other embodiments, the two or more vendors comprise food service providers operating separate kitchens.

In still other embodiments, at least a second floor of the one or more floors is occupied by a pharmacy.

In yet other embodiments, the one or more floors may comprise one or more horizontal transport systems for transporting the one or more goods horizontally within the one or more floors.

In other embodiments, the multi-vendor drive-through complex further comprises one or more consolidation stations on at least one floor of the one or more floors. The one or more consolidation stations consolidate the one or more goods into the orders.

In further accord with embodiments, the orders comprise two or more sub-orders associated with two or more vendors.

In other embodiments, the two or more sub-orders are fulfilled based on preparation times for the two or more sub-orders and order backlog of the two or more vendors.

In still other embodiments, the two or more sub-orders are further fulfilled based on a location of the user.

In yet other embodiments, the users comprise end users or one or more third party users that pick-up the orders placed by the one or more end users.

In other embodiments, the pick-up stations have compartments that are adjustable to provide the orders to the users in user vehicles at different heights.

In further accord with embodiments, at least one of the pick-up stations comprise at least a hot storage compartment and a cold storage compartment.

In other embodiments, the one or more drive-through lanes comprise two or more drive-through lanes and each of the two or more drive-through lanes have two or more pick-up stations.

Other embodiments of the present disclosure comprises a system for operating a multi-vendor drive-through complex, wherein the multi-vendor drive-through complex allows multiple vendors to provide goods to users. The system comprises at least one network communication interface, at least one non-transitory storage device, and at least one processing device coupled to the at least one non-transitory storage device and the at least one network communication interface. The at least one processing device is configured to receive orders from the users having one or more goods, consolidate and process the orders received from the users. The system further transmits the orders to vendors, wherein at least one order is transmitted to two or more vendors. The system determines that the orders are complete and operates one or more vertical transport systems to deliver the orders with the one or more goods to the pick-up stations. The users pick-up the orders from the pick-up stations via one or more drive-through lanes with the pick-up stations.

Another embodiment comprises a method for facilitating order placement, consolidation, and fulfillment of multi-vendor orders via a drive-through complex. The method comprises receiving an order from a user device of a user. The order comprises at least two sub-orders from at least two vendors. The system consolidates and processes the order received from the user. The system identifies preparation times associated with the at least two sub-orders and transmits the preparation times to the two or more vendors through the vendor systems. The system identifies a status of the at least two sub-orders and determines an available drive-through lane with an available pick-up station for the order with the at least two sub-orders. The system transmits one or more notifications to the user or a third party user associated with at least one of the status of the order and the available pick-up station to pick-up the order.

Other embodiments of the present disclosure comprise a multi-vendor drive-through complex for allowing multiple vendors to provide one or more goods to one or more users. The multi-vendor drive-through complex comprises one or more floors occupied by one or more vendors and one or more drive-through lanes located below the one or more floors for allowing the users to pick up orders from one or more pick-up stations. The complex further comprises a vertical transport system between the one or more floors and the one or more drive-through lanes. Moreover, the system uses one or more robotic delivery systems for delivering the orders from the one or more floors to the one or more pick up stations.

In other embodiments, the one or more robotic delivery systems comprise a drive assembly configured to move the robotic delivery system and an order delivery assembly configured to deliver an order to the one or more pick up stations.

In still other embodiments, the order delivery assembly comprises a vertical positioning portion for moving the order vertically within the robotic delivery system for delivery to the one or more pick-up stations.

In yet other embodiments, the order delivery assembly comprises a horizontal positioning portion for moving the order horizontally for delivery to the one or more pick up stations.

In other embodiments, the one or more robotic delivery systems are configured to move between the multiple vendors to receive orders from multiple vendors, move from the one or more floors to the one or more drive through lanes through the one or more vertical transport systems, and move to the one or more pick-up stations through a robotic delivery lane.

In further accord with other embodiments, the one or more robotic delivery systems are configured to move to one or more consolidation areas to receive orders from multiple vendors, move from the one or more floors to the one or more drive through lanes through the one or more vertical transport systems, and move to the one or more pick-up stations to deliver the orders through a robotic delivery lane.

In other embodiments, the one or more pick-up stations comprise an order receiving portion configured to receive the order from a robotic delivery system. The one or more pick-up stations may further comprise an order holding portion configured to hold the order until the user is identified as present and an order delivery portion configured to deliver an order to the user.

In still other embodiments, the one or more pick-up stations further comprise an order handling robot configured to pick an order from a robotic delivery system and deliver the order to the order holding portion.

Another embodiment of the present disclosure comprises a drive-through complex for providing services to users. The complex comprises a drive-through medical services section. The drive-through medical services section comprises a plurality of medical service stations configured to provide medical services to patients. The complex further comprises one or more floors located at least partially above or below the drive through medical services section having one or more vendors. The one or more vendors comprise a medical distribution vendor for providing medical orders of medical goods to the drive-through medical services section.

In other embodiments, the plurality of medical service stations comprise a plurality of patient rooms adjacent parking spaces.

In still other embodiments, the plurality of medical service stations comprise a plurality of drive-in bays.

In yet other embodiments, the plurality of drive-in bays comprise one or more open bays, and one or more privacy bays.

In other embodiments, the one or more open bays are utilized to initiate the medical services to the patients and the one or more privacy bays are utilized to provide private medical services to the patients.

In further accord with embodiments, the plurality of medical service stations comprise ventilation equipment for removing vehicle exhaust from adjacent the plurality of medical service stations.

In other embodiments, the complex further comprises a vertical transport system between the one or more floors and the drive-through medical services section.

In still other embodiments, the vertical transport system comprises a pneumatic delivery system configured to deliver the medical orders from the medical distribution vendor to the drive-through medical services section.

In yet other embodiments, the vertical transport system comprises one or more elevators and the complex further comprises one or more robotic delivery systems for delivering the medical orders from the medical distribution vendor on the one or more floors to the drive-through medical services section.

In other embodiments, the complex further comprises one or more medical pick-up stations configured to store the medical order for the patient users or third-party users.

In further accord with embodiments, the medical distribution vendor comprises at least a pharmacy.

In other embodiments, the one or more floors comprise a plurality of patient rooms for providing additional medical services to user.

In yet other embodiments, the one or more floors comprise a plurality of offices for medical professionals.

In still other embodiments, at least one floor of the one or more floors is occupied by multiple vendors and the complex comprises a drive-through pick-up section adjacent the drive-through medical section, wherein the drive-through pick-up section comprises one or more drive-through lanes for allowing user to pick-up vendor orders and one or more pick-up stations for storing one or more goods associated with the vendor orders.

In other embodiments, the one or more floors comprise a common area with one or more pick-up stations for receiving the vendor orders in the common area.

The features, functions, and advantages that have been discussed may be achieved independently in various embodiments of the present invention or may be combined with yet other embodiments, further details of which can be seen with reference to the following description and drawings.

### BRIEF DESCRIPTION OF THE DRAWINGS

Having thus described embodiments of the invention in general terms, reference will now be made the accompanying drawings, wherein:

FIG. 1 provides a block diagram illustrating a system environment for facilitating order placement, consolidation, and fulfillment of multi-vendor orders, in accordance with embodiments of the invention;

FIG. 2 provides a block diagram illustrating the vendor system 200 of FIG. 1, in accordance with embodiments of the invention;

FIG. 3 provides a block diagram illustrating a multi-vendor order fulfillment system 300 of FIG. 1, in accordance with embodiments of the invention;

FIG. 4 provides a block diagram illustrating the computing device system 400 of FIG. 1, in accordance with embodiments of the invention;

FIG. 5A illustrates a side elevation view of a multi-vendor drive-through complex, in accordance with embodiments of the invention;

FIG. 5B illustrates a front elevation view of a multi-vendor drive-through complex, in accordance with embodiments of the invention;

FIG. 5C illustrates a rear elevation view of a multi-vendor drive-through complex, in accordance with embodiments of the invention;

FIG. 6A illustrates a site plan for the first floor or ground floor of the multi-vendor drive-through complex, in accordance with embodiments of the invention;

FIG. 6B illustrates an enlarged view of a portion of the drive-through lanes of the multi-vendor drive-through complex, in accordance with embodiments of the invention;

FIG. 7A illustrates a site plan for an upper floor, such as a second floor, of the multi-vendor drive-through complex, in accordance with embodiments of the invention;

FIG. 7B illustrates an enlarged view of a ghost station present on an upper floor, such as a second floor of the multi-vendor drive-through complex, in accordance with embodiments of the invention;

FIG. 8 illustrates an enlarged view of the site plan for an upper floor, such as a third floor, of the multi-vendor drive-through complex, in accordance with embodiments of the invention;

FIG. 9A illustrates a site plan for an upper floor, such as a fourth floor, of the multi-vendor drive-through complex, in accordance with embodiments of the invention;

FIG. 9B illustrates a site plan for an upper floor, such as a roof, of the multi-vendor drive-through complex, in accordance with embodiments of the invention;

FIG. 10 illustrates a flowchart for a process flow for facilitating order placement, consolidation, and fulfillment of multi-vendor orders, in accordance with embodiments of the invention;

FIG. 11 illustrates a block diagram of a robotic delivery system for delivering multi-vendor orders from one or more floors to one or more pick-up stations, in accordance with embodiments of the invention;

FIG. 12 illustrates a block diagram of a pickup-station for storing multi-vendor orders, in accordance with embodiments of the invention; and

FIG. 13 illustrates a process flow for fulfilling orders using the applications and systems, in accordance with embodiments of the invention.

FIG. 14 illustrates a schematic of a first or ground floor in which staging areas, access areas, and/or medical service stations may be provided, in accordance with embodiments of the invention.

FIG. 15 illustrates a schematic of a first or ground floor in which staging areas, access areas, and/or medical service stations may be provided, in accordance with embodiments of the invention.

FIG. 16 illustrates a schematic of a first or ground floor in which staging areas, access areas, and/or medical service stations may be provided, in accordance with embodiments of the invention.

FIG. 17 illustrates traffic flow for the multi-vendor complex, in accordance with embodiments of the invention.

FIG. 18 illustrates a front elevation view of a multi-vendor complex with a garage and medical service stations, in accordance with embodiments of the invention.

FIG. 19A illustrates traffic flow for the multi-vendor complex and a floor of the garage, in accordance with embodiments of the invention.

FIG. 19B illustrates an enlarged view of the traffic flow for the multi-vendor complex and a floor of the garage, in accordance with embodiments of the invention.

FIG. 20 illustrates a high level process flow for routing patients to different floors of the garage of the multi-vendor complex, in accordance with embodiments of the invention.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

In accordance with embodiments of the invention, the term “vendors” may include any business organization that provides goods and/or services to one or more customers. Examples of vendors may include, but are not limited to, restaurants, fast food, fast casual, ghost kitchens, pharmacies, e-commerce institutions, clothing stores, hardware stores, home improvement stores, grocery stores, retail warehouses, convenience stores, electronic stores, lens/glasses stores, liquor stores, any type of retail store, and/or the like.

Furthermore, embodiments of the present invention use the term “user,” “end user,” or “customer user.” In accordance with embodiments of the invention, a user may be a customer that utilizes the present invention to place one or more multi-vendor orders associated with one or more goods provided by one or more vendors located in the multi-vendor drive-through complex. In accordance with embodiments of the invention, the terms “third party” may include any delivery company that delivers one or more goods to users. The term “third party user” may be an employee of the delivery company that picks up one or more goods from the multi-vendor drive-through complex and delivers them to end users.

Many of the example embodiments and implementations described herein contemplate interactions engaged in by a user with a computing device and/or one or more communication devices and/or secondary communication devices. Furthermore, as used herein, the term “user computing device” or “mobile device” may refer to mobile phones, computing devices, tablet computers, wearable devices, smart devices and/or any portable/non-portable electronic device capable of receiving and/or storing data therein.

A “user interface” is any device or software that allows a user to input information, such as commands or data, into a device, or that allows the device to output information to the user. For example, the user interface include a graphical user interface (GUI) or an interface to input computer-executable instructions that direct a processing device to carry out specific functions. The user interface typically employs certain input and output devices to input data received from a user or output data to a user. These input and output devices may include a display, mouse, keyboard, button, touchpad, touch screen, microphone, speaker, LED, light, joystick, switch, buzzer, bell, and/or other user input/output devices for communicating with one or more users.

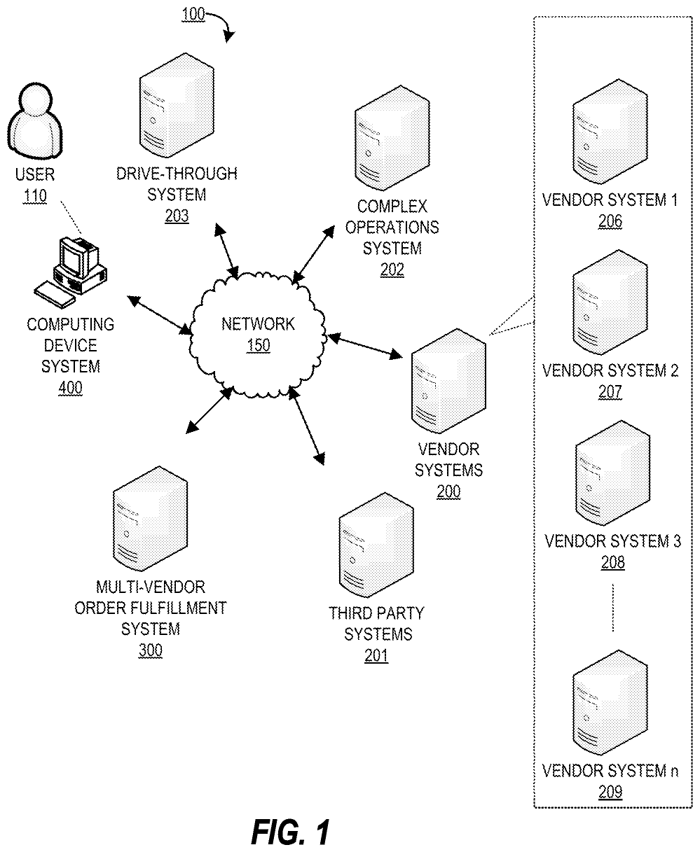

FIG. 1 provides a block diagram illustrating a system environment 100 for facilitating order placement, consolidation, and fulfillment of multi-vendor orders, in accordance with embodiments of the invention. As illustrated in FIG. 1, the environment 100 includes a multi-vendor order fulfillment system 300, one or more vendor systems 200, a computing device system 400, third party systems 201, a drive through system 203, and a complex operations system 202. One or more users 110 may be included in the system environment 100, where the users 110 interact with the other systems of the system environment 100 via a user interface of the computing device system 400 (e.g., user computer system, or the like). The one or more users 110 may be end users who place one or more orders associated with goods provided by one or more vendors associated with the one or more vendor systems 200, where the one or more orders may be placed from a location that does not match the location of the multi-vendor drive-through complex (e.g., end user's workplace, home, apartment, vehicle, mobile location, or the like).

The vendor systems 200 may be any systems owned or otherwise controlled by a vendor to support or perform one or more process steps described herein. In some embodiments, the vendor systems 200 may comprise one or more independent systems (e.g., vendor system ‘1’ 206, vendor system ‘2’ 207, vendor system ‘3’ 208, through vendor system ‘n’ 209) that provide one or more goods to one or more users 110. In an exemplary embodiment, vendor system ‘1’ 206 may be associated with a fast food restaurant that has a ghost station (e.g., ghost kitchen) in the multi-vendor drive-through complex, vendor system ‘2’ 207 may be associated with a bakery that has a ghost station (e.g., ghost kitchen) in the multi-vendor drive-through complex, vendor system ‘3’ 208 may be associated with a pharmacy, and vendor system ‘n’ 209 may be associated with an e-commerce warehouse. One or more vendor systems may be associated with, but are not limited to any of the vendors described herein. Moreover, while this specification discusses the combination of ghost kitchens on the first floor (e.g., first vendor floor above the drive-through lanes on the ground floor) and a pharmacy on the second vendor floor, it should be understood that any of these vendors may be located on any floor (e.g., any floor of a building having two or more floors), and in any combination on a floor (e.g., one or more vendors on a single floor).

Third party systems 201 may be any systems that provide delivery services to the one or more users 110, where third party users associated with the third party systems 201 pick up one or more goods associated with one or more orders placed by one or more users 110 from the multi-vendor drive-through complex and deliver to the one or more users 110. The third party systems 201, may include maned delivery systems, autonomous ground vehicle systems, drone systems, or the like.

Drive through system 203 may be a system that operates by communicating to other systems within the environment 100 to control and/or manage one or more pick-up stations (e.g., kiosks, human interface stations, lockers, or the like) present in the one or more drive-through lanes or other locations in the multi-vendor complex. In some embodiments, the drive-through system 203 may be a part of the multi-vendor fulfillment system 300.

Complex operations system 202 may be any system that controls one or more robotic delivery systems 1100, one or more vertical transport systems (e.g., one or more dumbwaiters, one or more elevators, or the like), one or more horizonal transport systems (e.g., conveyors, tracks, or the like), one or more pick up stations, one or more building equipment systems (e.g., air conditioners, freezers, fryers, conveyer systems, and/or the like), and/or the like. In some embodiments, the complex operations system 202 may be a part of the multi-vendor order fulfillment system 300. In particular, with respect to the robotic delivery systems 1100, the complex operation systems 202 may communicate with the robotic delivery systems 1100 in order to control the movements and distribution of the orders to and from the robotic delivery systems 1100, as will be discussed in further detail herein.

The multi-vendor order fulfillment system 300 is a system of the present invention for performing one or more process steps described herein. The multi-vendor order fulfillment system 300, the vendor system 200, the computing device system 400, the drive through system 203, the complex operations system 202, and/or the third party systems 201 may be in network communication across the system environment 100 through the network 150. The network 150 may include a local area network (LAN), a wide area network (WAN), and/or a global area network (GAN). In some embodiments, the multi-vendor order fulfillment system 300, the vendor system 200, the computing device system 400, the drive through system 203, the complex operations system 202, and/or the third party systems 201 may communicate via a network established via Bluetooth communication, RFID, near-field communication (NFC), mesh networking (e.g., Zigbee, or the like), scanners, or any other forms of wireless communications. The network 150 may provide for wireline, wireless, or a combination of wireline and wireless communication between devices in the network. In one embodiment, the network 150 includes the Internet. In general, the multi-vendor order fulfillment system 300 is configured to communicate information or instructions to, or receive information or instructions from, the vendor system 200, the computing device system 400, the drive through system 203, the complex operations system 202, and/or the third party systems 201 across the network 150.

The computing device system 400 may be a system owned or controlled by the entity of the vendor system 200 and/or the user 110. As such, the computing device system 400 may be a computing device of the user 110 (e.g., a mobile device as will be discussed herein, such as a smartphone, wearable device, or the like). In general, the computing device system 400 communicates with the user 110 via a user interface of the computing device system 400, and in turn is configured to communicate information or instructions to, or receive information or instructions from, the multi-vendor order fulfillment system 300, vendor system 200, the drive through system 203, the complex operations system 202, and/or the third party systems 201 across the network 150. While the vendor systems 200, third-party systems 202, complex operations system 202, drive-through system 203, multi-vendor order fulfillment system 300, and/or the like are illustrated a separate systems, it should be understood that one or more of these systems, and the functions thereof, may be combined into a single system.

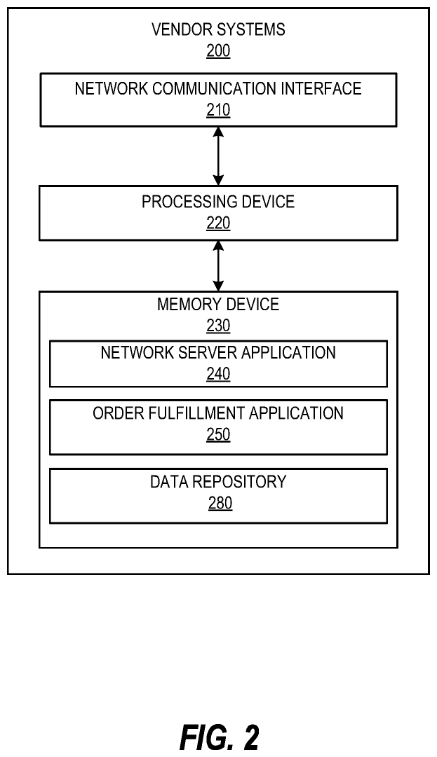

FIG. 2 provides a block diagram illustrating the vendor system 200, in greater detail, in accordance with embodiments of the invention. As illustrated in FIG. 2, in one embodiment of the invention, the vendor system 200 includes one or more processing devices 220 operatively coupled to a network communication interface 210 and a memory device 230.

It should be understood that the memory device 230 may include one or more databases or other data structures/repositories. The memory device 230 also includes computer-executable program code that instructs the processing device 220 to operate the network communication interface 210 to perform certain communication functions of the vendor system 200 described herein. For example, in one embodiment of the vendor system 200, the memory device 230 includes, but is not limited to, an order fulfillment application 250, and a data repository 280. The computer-executable program code of the network server application 240 and the order fulfillment application 250 to perform certain logic, data-extraction, and data-storing functions of the vendor system 200 described herein, as well as communication functions of the vendor system 200.

The network server application 240 and the order fulfillment application 250 are configured to store data in the data repository 280 or to use the data stored in the data repository 280 when communicating through the network communication interface 210 with the multi-vendor order fulfillment system 300, the computing device system 400, and/or the third party systems 201 to perform one or more process steps described herein. In some embodiments, the data repository 280 comprises information associated with one or more resources of the entity. In some embodiments, the vendor system 200 may receive instructions from the multi-vendor order fulfillment system 300 via the order fulfillment application 250 to perform certain operations. The order fulfillment application 250 may be provided by the multi-vendor order fulfillment system 300, and as such the order fulfillment application 250 be located on multi-vendor order fulfillment system 300, a portion may be located on the multi-vendor order fulfillment system 300 and a portion may be located on the vendor systems 200, or the order fulfillment application 250 may communicate with the multi-vendor order fulfillment system 300.

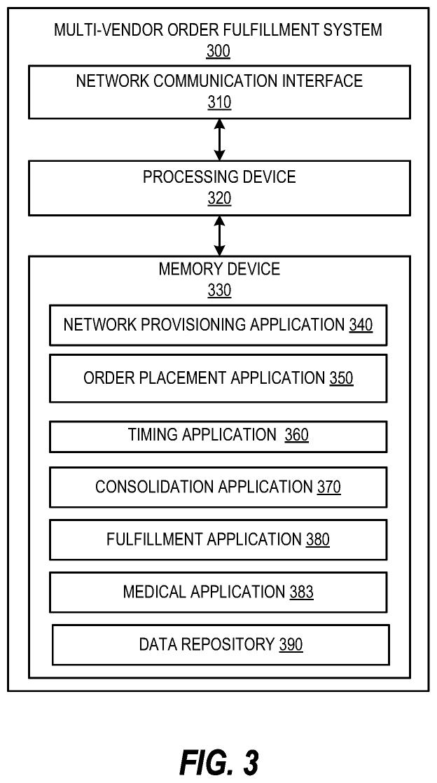

FIG. 3 provides a block diagram illustrating the multi-vendor order fulfillment system 300 in greater detail, in accordance with embodiments of the invention. As illustrated in FIG. 3, in one embodiment of the invention, the multi-vendor order fulfillment system 300 includes one or more processing devices 320 operatively coupled to a network communication interface 310 and a memory device 330.

It should be understood that the memory device 330 may include one or more databases or other data structures/repositories. The memory device 330 also includes computer-executable program code that instructs the processing device 320 to operate the network communication interface 310 to perform certain communication functions of the multi-vendor order fulfillment system 300 described herein. For example, in one embodiment of the multi-vendor order fulfillment system 300, the memory device 330 includes, but is not limited to, a network provisioning application 340, an order placement application 350, a consolidation application 370, a fulfillment application 380, a medical application 383, and a data repository 390 comprising data processed or accessed by one or more applications in the memory device 330. The computer-executable program code of the network provisioning application 340, the order placement application 350, the consolidation application 370, the fulfillment application 380, and the medical application 383 may instruct the processing device 320 to perform certain logic, data-processing, and data-storing functions of the multi-vendor order fulfillment system 300 described herein, as well as communication functions of the multi-vendor order fulfillment system 300.

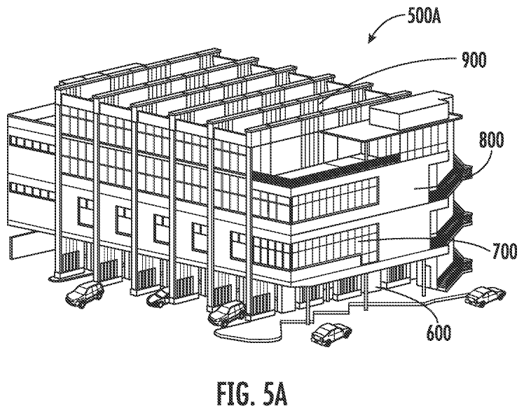

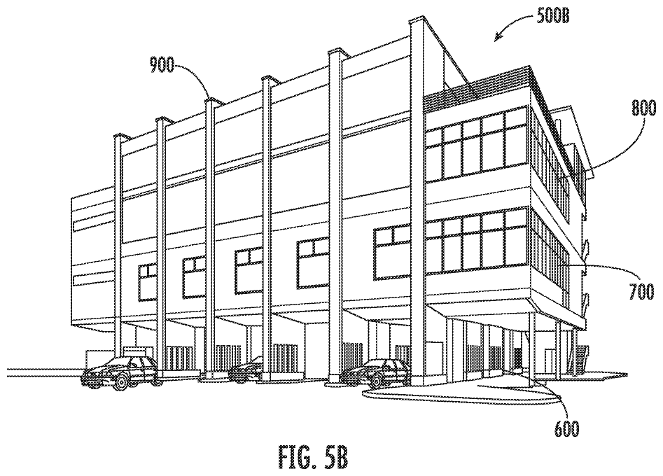

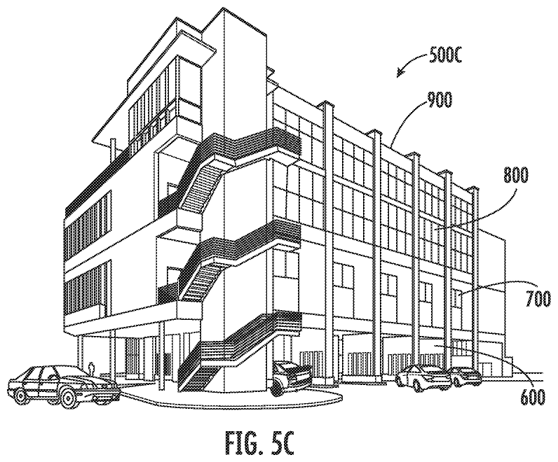

The network provisioning application 340, the order placement application 350, the consolidation application 370, the fulfillment application 380, and the medical application 383 are configured to invoke or use the data in the data repository 390 when communicating through the network communication interface 310 with the vendor system 200, the computing device system 400, and/or the third party systems 201. In some embodiments, the network provisioning application 340, the order placement application 350, the consolidation application 370, the fulfillment application 380, and the medical application 383 may store the data extracted or received from the vendor system 200, the third party system 120, and the computing device system 400 in the data repository 390. In some embodiments, the network provisioning application 340, the order placement application 350, the consolidation application 370, the fulfillment application 380, and the medical application 383 may be a part of a single application. The order placement application 350 allows the one or more users 110 to place one or more multi-vendor orders via the computing device system 400 via a part of the order placement application that is present in the computing device system 400. The consolidation application 370 processes sub-orders in a single order submitted by a user and provides one or more instructions to one or more vendors that are associated with each sub-order within the order placed by the user. The timing application 360 calculates the time taken to fulfill each of the sub-order based on real-time order backlog associated with the one or more vendors, location of the user, type of each of the sub-order, preparation time associated with each of the sub-order, and/or the like. The fulfillment application 380 may track the progress of the order and may assign a robotic delivery system 1100, drive-through lane and/or pick-up stations 622 (e.g., a kiosk, locker, or the like) for the user to pick-up the order based on the type of order, availability of the robotic delivery systems 1100 (e.g., location, hot/cold/room-temp/ambient-temp compartments available, current order on-board and queued for pick-up, the pick-up stations 622 where the orders will be delivered, or the like of the plurality of robotic delivery systems 1100), and/or the pick-up stations 622 (e.g., kiosks that are available, features of the kiosks, such as size, hot/cold/room-temp/ambient temp compartments, location, delivery lane 601, autonomous delivery lane, bike, scooter, walk-up location, drone location, or the like). In some embodiments, the fulfillment application 380 may also transmit one or more control signals/instructions to the vendor systems 200, drive-through system 203, and the complex operations system 202. In some embodiments, the fulfillment application 380 may transmit one or more notifications about the order progress to the user, a third party user, and/or the one or more vendors processing the order. The medical application 383 may allow a user (e.g., patient, caregiver, medical professional either off-site or on-site) to provide or receive medical information (e.g., symptoms, aliments, medical issues, insurance information, medical history, primary and specialist information, or the like) so that a decisioning action may be made regarding where to direct the user within the complex. The functionalities provided by the order placement application 350, the consolidation application 370, the fulfillment application 380, and the medical application 383 are explained in a greater detail in FIG. 5 through FIG. 20.

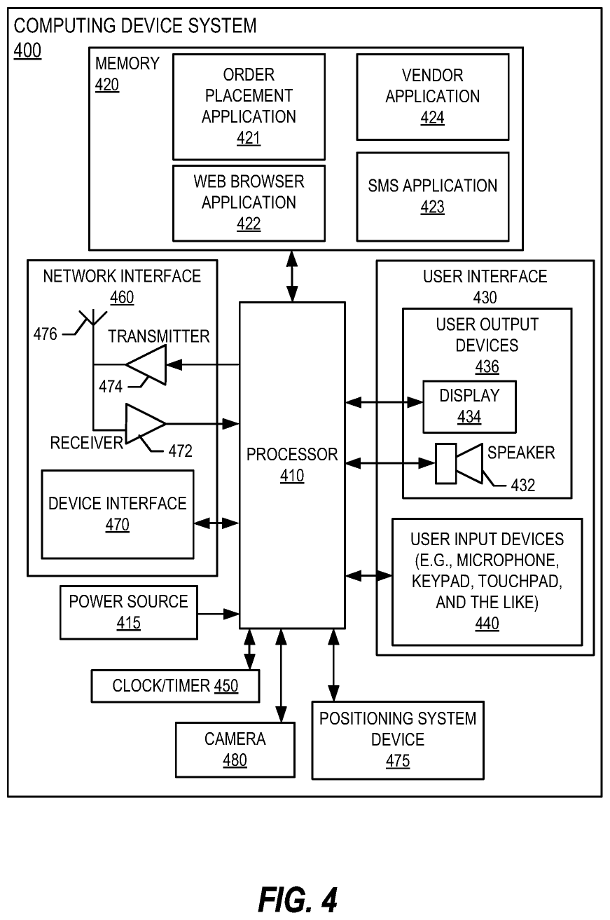

FIG. 4 provides a block diagram illustrating a computing device system 400 of FIG. 1 in more detail when the computing device system 400 is a user computing device system 400. As such, the user computing device system 400 may be a desktop, laptop or mobile computing device (e.g., smartphone, wearable device, or the like). However, it should be understood that a mobile smartphone is merely illustrative of one type of computing device system 400 that may benefit from, employ, or otherwise be involved with embodiments of the present invention and, therefore, should not be taken to limit the scope of embodiments of the present invention. Other types of computing devices may include portable digital assistants (PDAs), pagers, mobile televisions, gaming devices, desktop computers, workstations, laptop computers, cameras, video recorders, audio/video player, radio, GPS devices, wearable devices (e.g., watches, glasses, implants, or the like), Internet-of-things devices, augmented reality devices, virtual reality devices, automated teller machine devices, electronic kiosk devices, or the like, or any combination of the aforementioned.

Some embodiments of the computing device system 400 include a processor 410 communicably coupled to such devices as a memory 420, user output devices 436, user input devices 440, a network interface 460, a power source 415, a clock or other timer 450, a camera 480, and a positioning system device 475. The processor 410, and other processors described herein, generally include circuitry for implementing communication and/or logic functions of the computing device system 400. For example, the processor 410 may include a digital signal processor device, a microprocessor device, and various analog to digital converters, digital to analog converters, and/or other support circuits. Control and signal processing functions of the computing device system 400 are allocated between these devices according to their respective capabilities. The processor 410 thus may also include the functionality to encode and interleave messages and data prior to modulation and transmission. The processor 410 can additionally include an internal data modem. Further, the processor 410 may include functionality to operate one or more software programs, which may be stored in the memory 420. For example, the processor 410 may be capable of operating a connectivity program, such as a web browser application 422 (or a dedicated application, such as an applet, or the like). The web browser application 422 may allow the computing device system 400 to transmit and receive web content, such as, for example, location-based content and/or other web page content, according to a Wireless Application Protocol (WAP), Hypertext Transfer Protocol (HTTP), and/or the like.

The processor 410 is configured to use the network interface 460 to communicate with one or more other devices on the network 150. In this regard, the network interface 460 includes an antenna 476 operatively coupled to a transmitter 474 and a receiver 472 (together a “transceiver”). The processor 410 is configured to provide signals to and receive signals from the transmitter 474 and receiver 472, respectively. The signals may include signaling information in accordance with the air interface standard of the applicable cellular system of the wireless network 152. In this regard, the computing device system 400 may be configured to operate with one or more air interface standards, communication protocols, modulation types, and access types. By way of illustration, the computing device system 400 may be configured to operate in accordance with any of a number of first, second, third, fourth, fifth, six, or the like generation communication protocols and/or the like.

As described above, the computing device system 400 has a user interface that is, like other user interfaces described herein, made up of user output devices 436 and/or user input devices 440. The user output devices 436 include a display 430 (e.g., a liquid crystal display or the like) and a speaker 432 or other audio device, which are operatively coupled to the processor 410.

The user input devices 440, which allow the computing device system 400 to receive data from a user such as the user 110, may include any of a number of devices allowing the computing device system 400 to receive data from the user 110, such as a keypad, keyboard, touch-screen, touchpad, microphone, mouse, joystick, other pointer device, button, soft key, and/or other input device(s). The user interface may also include a camera 480, such as a digital camera. In some embodiments, the computing device system 400 may comprise facial recognition, GPS recognition, retina recognition capabilities, or the like, where such capabilities may allow the system of the present invention to recognize the presence of the user via the vendor application 424 and/or order placement application 421.

The computing device system 400 may also include a positioning system device 475 that is configured to be used by a positioning system to determine a location of the computing device system 400. For example, the positioning system device 475 may include a GPS transceiver. In some embodiments, the positioning system device 475 is at least partially made up of the antenna 476, transmitter 474, and receiver 472 described above. For example, in one embodiment, triangulation of cellular signals may be used to identify the approximate or exact geographical location of the computing device system 400. In other embodiments, the positioning system device 475 includes a proximity sensor or transmitter, such as an RFID tag, near-field communication, mesh network, Zigbee communication, and/or other wireless communication, that can sense or be sensed by devices known to be located proximate a building, drive through lane, pick-up station, or other location to determine that the computing device system 400 is located proximate these known devices.

The computing device system 400 further includes a power source 415, such as a battery, for powering various circuits and other devices that are used to operate the computing device system 400. Embodiments of the computing device system 400 may also include a clock or other timer 450 configured to determine and, in some cases, communicate actual or relative time to the processor 410 or one or more other devices.

The computing device system 400 also includes a memory 420 operatively coupled to the processor 410. As used herein, memory includes any computer readable medium (as defined herein below) configured to store data, code, or other information. The memory 420 may include volatile memory, such as volatile Random Access Memory (RAM) including a cache area for the temporary storage of data. The memory 420 may also include non-volatile memory, which can be embedded and/or may be removable. The non-volatile memory can additionally or alternatively include an electrically erasable programmable read-only memory (EEPROM), flash memory or the like.

The memory 420 can store any of a number of applications which comprise computer-executable instructions/code executed by the processor 410 to implement the functions of the computing device system 400 and/or one or more of the process/method steps described herein. For example, the memory 420 may include such applications as a conventional web browser application 422, an order placement application 421, a vendor application 424. These applications also typically instructions to a graphical user interface (GUI) on the display 430 that allows the user 110 to interact with the vendor system 200, the multi-vendor order fulfillment system 300, and/or other devices or systems. The memory 420 of the computing device system 400 may comprise a Short Message Service (SMS) application 423 configured to send, receive, and store data, information, communications, alerts, and the like via the network 150, such as a wireless telephone network 152. In some embodiments, the order placement application 421 provided by the multi-vendor order fulfillment system 300 allows the user 110 to access the multi-vendor order fulfillment system 300. In some embodiments, the vendor application 424 provided by the vendor system 200 and the order placement application 421 provided by the multi-vendor order fulfillment system 300 allow the user 110 to access the functionalities provided by the multi-vendor order fulfillment system 300 and the vendor system 200.

The memory 420 can also store any of a number of pieces of information, and data, used by the computing device system 400 and the applications and devices that make up the computing device system 400 or are in communication with the computing device system 400 to implement the functions of the computing device system 400 and/or the other systems described herein.

Turning to the components of the multi-vendor drive-through complex, FIG. 5A illustrates a side elevation view 500A of the multi-vendor drive-through complex, in accordance with embodiments of the invention. Moreover, FIG. 5B illustrates a front elevation view 500B of a multi-vendor drive-through complex, in accordance with embodiments of the invention. Furthermore, FIG. 5C illustrates a rear elevation view 500C of a multi-vendor drive-through complex, in accordance with embodiments of the invention. The multi-vendor drive-through complex may comprise ‘n’ number of floors, where the ‘n’ number of floors are occupied by one or more vendors. As shown, the multi-vendor drive-through complex, in some embodiments, may comprise a first floor 600, a second floor 700, a third floor 800, and a roof. The number of floors, and the numbering of the floors, shown in the figures are for illustrative purposes only. In some embodiments, each of the ‘n’ number of floors may be occupied by a different vendor. For example, the second floor 700 may be occupied by one or more restaurants (e.g., ghost kitchens, or the like), the third floor 800 may be occupied by a pharmacy, a fourth floor (not shown) may be occupied by an e-commerce warehouse, and the like. In another example, the fourth floor may be a mezzanine level comprising storage areas, HVAC units, maintenance areas, drone pick-up areas, or the like. The multi-vendor drive-through complex may further comprise a roof that allows one or more drones to pick up one or more orders and the roof may further comprise HVAC units, exhaust hoods, roof drainage systems, and/or the like. In some embodiments, a single floor of the ‘n’ number of floors may be occupied by multiple vendors associated with same category of goods. For example, the second floor 700 may comprise one or more ghost stations (e.g., ghost kitchens) that may be occupied by multiple vendors (e.g., based on cuisine, type of food, etc.). In some embodiments, each of the ‘n’ number of floors may be structured to have walls that are made up of glass or any other similar material such that the interior of the multi-vendor drive-through complex is visible to the one or more users and/or third party users driving in to pick up one or more orders. In some embodiments, the multi-vendor drive through complex may comprise vertical transport systems between one or more floors, such as elevators (e.g., sized for the robotic delivery systems, sized for humans, or sized for combinations thereof), dumb-waiters, or other like systems that transports products (e.g., for orders) as will be described in further detail herein. Each of the floors may also comprise horizonal transport systems, such as conveyers, tracks (e.g., overhead, floor, or the like), and/or containers that allow products from one or more vendors to be consolidated and/or moved to consolidation areas, and be transported to the ground level, floors, and/or roof through the vertical transport systems and/or through the use of robotic delivery systems 1100, which will be discussed in further detail herein.

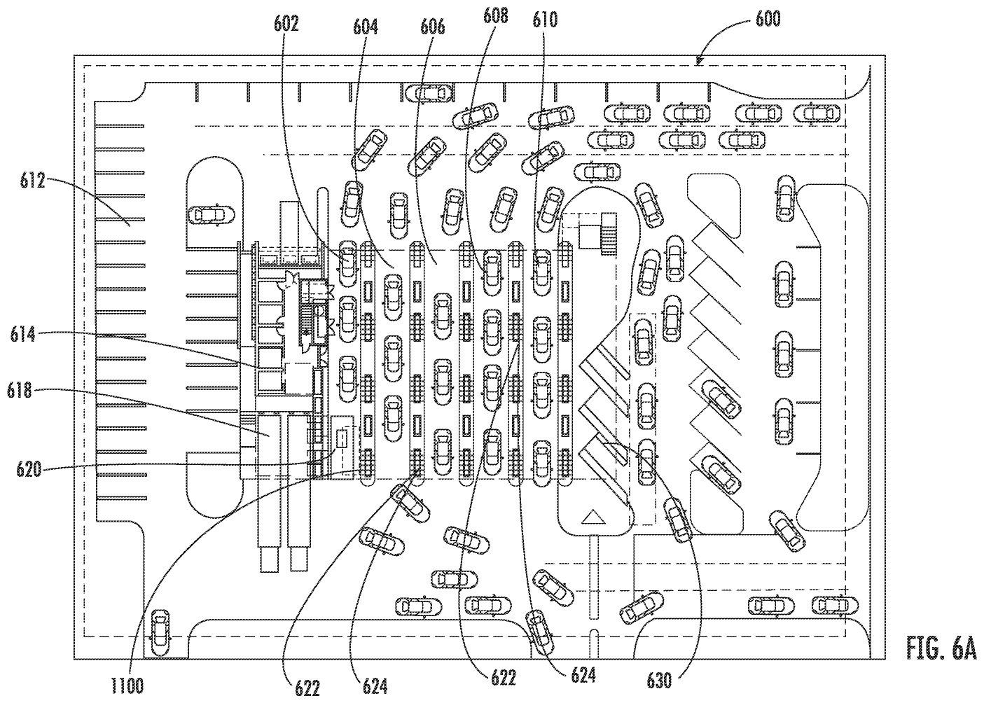

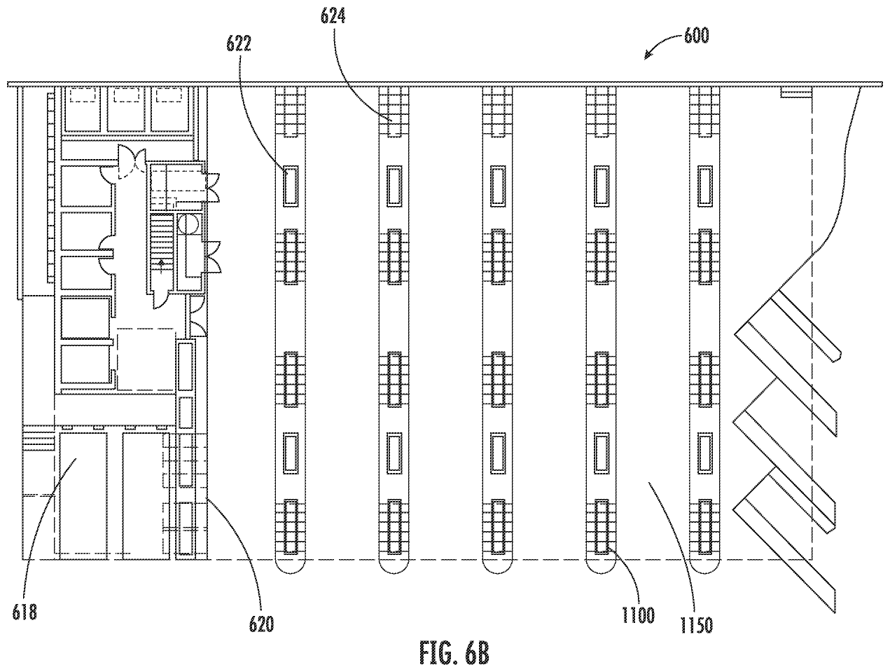

FIG. 6A illustrates a site plan for the first floor (e.g., ground floor) of the multi-vendor drive-through complex 600, in accordance with embodiments of the invention. While the drive-through lanes may be provided on a ground floor of the multi-vendor drive-through complex 600, it should be understood that the drive-through lanes may be located below the ground level (e.g., one or more levels down) or above the ground level (e.g., one or more levels up) depending on the site and configuration of the complex 600. FIG. 6B illustrates an enlarged view of the drive-through lanes of the multi-vendor drive-through complex, in accordance with embodiments of the invention. As shown, the first floor of the multi-vendor drive-through complex 600 may comprise one or more drive-through lanes 601 (e.g., 602, 604, 606, 608, 610) for one or more users and/or third party users to pick up goods associated with one or more orders. Each of the one or more drive-through lanes 601 may comprise one or more pick-up stations 622 (e.g., one or more kiosks, one or more pick-up boxes, one or more dumbwaiters, one or more handling and storage robots, or the like). In some embodiments, one or more lanes of the one or more drive-through lanes 601 may be bypass lanes, where one or more users and/or third party users who have already picked-up the orders can exit a drive-through lane 601 using the bypass lanes without having to wait behind other users. In some embodiments, each of the one or more drive-through lanes 601 may be wide enough to allow two vehicles to pass through, thereby minimizing the wait times. In alternate embodiments, the pick-up stations 622 may be spaced apart such that vehicles may pass between adjacent drive through lanes 601. In some embodiments, there may be separate drive-through lanes and/or specific pick-up stations for autonomous delivery vehicles. The autonomous delivery vehicles may be loaded by humans; however, in some embodiments a material handling robot may be used for placing orders in the autonomous delivery vehicle. In other embodiments, there may be separate lanes and/or locations at which users on other modes of transportation (e.g., bicycles, scooters, or the like manual or electric powered transportation) may pick-up orders from pick-up stations away from the user vehicles (e.g., combustion, electric, or the like cars, trucks, or the like). As shown, in some embodiments, each drive-through lane may comprise one or more vertical transport systems (e.g., dumbwaiters 624, elevators, or the like) that deliver orders to one or more pick-up stations 622 (e.g., located below the dumbwaiters, adjacent the dumbwaiters, or the like).

In some embodiments, the pick-up stations 622 may have multiple compartments at different heights, and/or compartments that can be adjusted, such that the entire compartment or a shelf within the pick-up station 622 can be adjusted based on the type of vehicle (e.g., location of window of an SUV, sedan, sports car, or the like) in which the user is located. As such, the pick-up station 622 may automatically adjust the vertical location of the compartment based on the height of the access for the vehicle (e.g., the height of the window of the car based on the vehicle size). In some embodiments, two drive-through lanes may be back to back, where either side of a pick-up station 622 (e.g., kiosk, locker, pick-up box, a handling and storage robot, and/or a dumbwaiter) may be utilized by users in the two drive-through lanes. In some embodiments, each of the one or more pick-up stations 622 (e.g., kiosks, lockers, or the like) may comprise a digital screen that allows the users picking up the orders to interact with one or more vendors (e.g., pharmacist, fast food restaurant employee, retail store employee, help desk employee, or the like) in real-time. In some embodiments, the system may project one or more advertisements, a list of vendors in the multi-vendor drive-through complex, live interaction feature with the one or more vendors, instructions associated with the one or more pick-up stations 622 (e.g., kiosks, lockers, or the like) and/or one or more drive-through lanes, welcome messages, thank you messages, one or more announcements, or the like on the digital screen. In some embodiments, the digital screen may be incorporated into the pick-up stations 622 and/or be independent from the one or more pick-up stations 622.

In some embodiments, when robotic delivery systems 1100 are utilized, robotic delivery lanes may be located adjacent the one or more pick-up stations 622. The robotic delivery lanes may be sized specifically for the robotic delivery systems 1100, which will be described in further detail herein. The robotic delivery lanes may be located between two rows of one or more pick-up stations 622 that are faced back to back. In some embodiments, one or more robotic delivery lanes may be substituted for the drive-through lanes 601 (e.g., 602, 604, 606, 608, 610) illustrated in the figures. The robotic delivery lanes may be enclosed such that users and/or user vehicles cannot interact with the robotic delivery systems 1100. For example, the robotic delivery lanes may be enclosed with glass and/or portions of glass, such that users may see the operation of the robotic delivery systems 1100 in the robotic delivery lanes and/or with the pick-up stations 622. The robotic delivery lanes may include at least one vertical transport system that allows the one or more robotic delivery systems 1100 to move between one or more floors to the one or more robotic delivery lanes and to interact with the pick-up stations 622, as will be described in further detail herein.

As shown, in some embodiments, additional parking spaces 612 may be present that allow one or more customer users and/or one or more third party users to wait to pick-up an order (e.g., when there is an error in the order, when part of the order is not ready, or the like). In some embodiments, there may be additional parking spaces that allow users to interact directly with personnel associated with one or more vendors. In some embodiments, ADA parking spaces 630 with pick-up stations may be present. As shown, one or more freight elevators 614 may be present to allow loading and/or unloading one or more goods. Delivery vehicles (e.g., trucks, or the like) may deliver the goods to a loading dock 618 adjacent the freight elevators 614. As shown, a separate space 618 may be allocated for one or more pick-up trucks to pick up bulk orders and/or unload one or more goods. In some embodiments, a bulk terminal 620 may be present allowing for delivery of bulk items (e.g., multiple kiosks for mobile delivery, a delivery truck to deliver to multiple orders to an apartment complex, event, or the like).

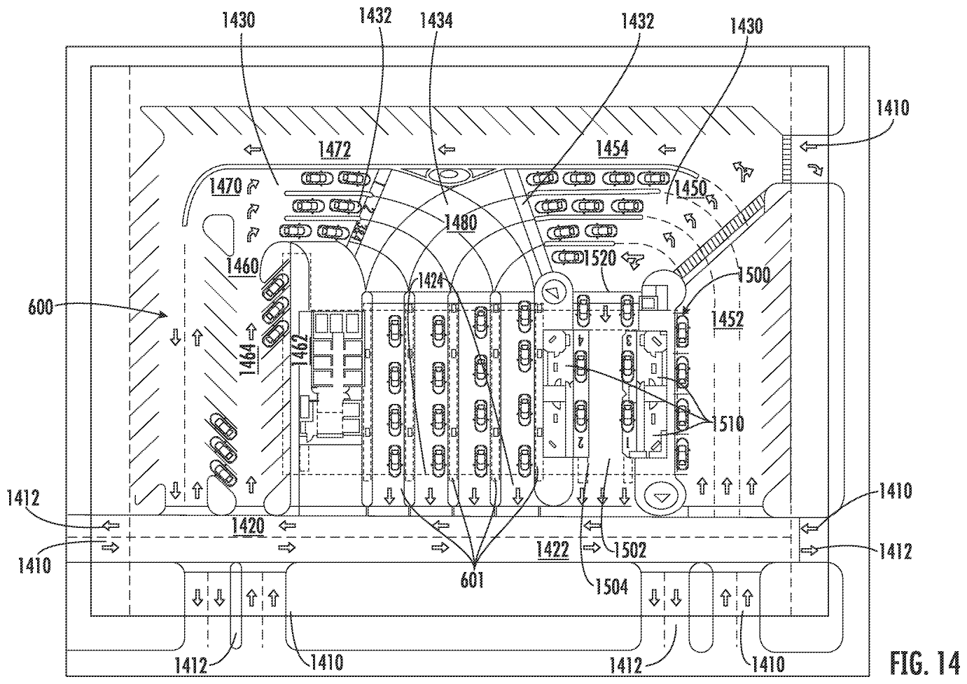

Moreover, due to the traffic that may be running through the complex at times, the complex may have additional lanes and traffic control elements, which may communicate with and/or work independently from or in conjunction with traffic control elements (e.g., lights, signs, merging, or the like) in the surrounding streets (e.g., corner streets, multiple streets surrounding a portion or all of the complex, or the like). As such, the complex may control the timing of when user (e.g., user vehicles, bicycle, scooter, lanes) and/or autonomous delivery vehicles enter and/or leave the complex. For example, as users and/or autonomous delivery vehicles enter the property of the complex, the drive-through systems 203 may hold the vehicles in staging areas before the vehicles are directed to particular lanes and/or pick-up stations. The flow of vehicles can be sped up or slowed down in the staging areas, in the lanes, and/or in access lanes to control the flow of vehicles entering, located within, and/or leaving the property. The movement of the vehicles may be based at least in part based on information received from surrounding traffic systems (e.g., lights, cameras, sensors, or the like) to determine when traffic is clear/reduced and/or heavy/backed-up on the surrounding streets. Embodiments of the flow of traffic will be discussed in further detail later with respect to FIG. 14.

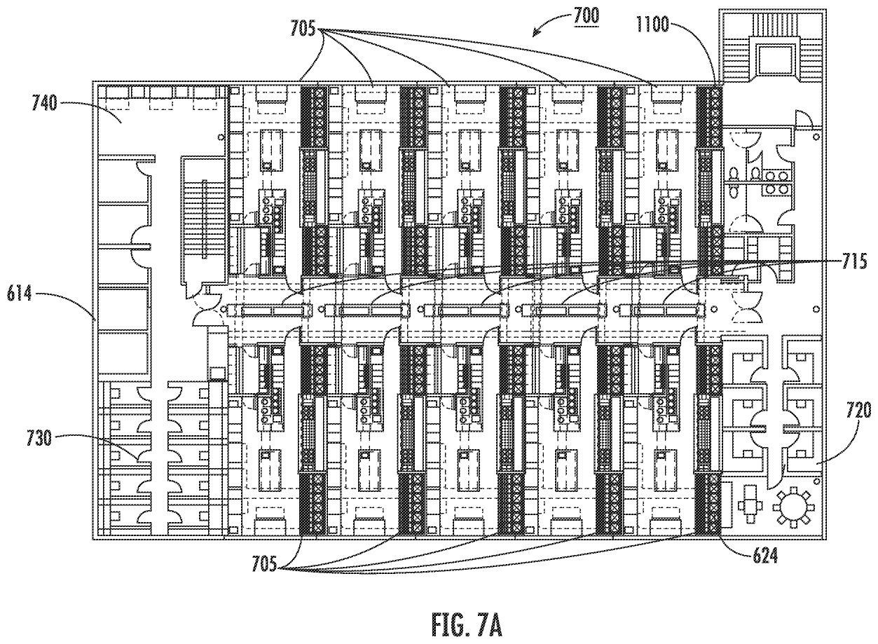

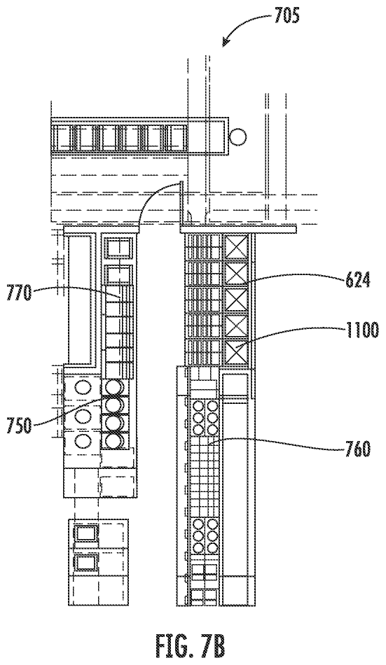

FIG. 7A illustrates a site plan for the second floor 700 (or other floor) of the multi-vendor drive-through complex, in accordance with embodiments of the invention. As shown, the second floor 700 may be occupied by one or more vendors that serve food, where the site plan for the second floor 700 comprises one or more ghost stations 705 (e.g., ghost kitchens) occupied by the one or more vendors. Each of the one or more ghost stations 705 may be configured based on one or more requirements of the vendors occupying the one or more ghost stations 705. As shown, one or more order consolidation areas 715 may be present, where one or more sub-orders associated with an order are combined. For example, a single order may comprise multiple sub-orders (e.g., appetizer, dessert, different cuisines, or the like) served by multiple vendors. In some embodiments, the one or more ghost stations 705 and the one or more consolidation areas 715 may be connected via a horizontal transport system (e.g., conveyer system, track system, or the like) that allows products for orders to be moved between vendors and/or consolidation areas 715 such that one or more employees associated with the vendors and/or one or more robotic delivery systems 1100 can consolidate one or more sub-orders associated with an order. The site plan associated with the second floor 700 may further comprise a break room and/or office space for the vendor 720, one or more secure individual or communal storage spaces 730 (e.g., storerooms, freezer space, or the like). In some embodiments, a designated place may be present in the site plan for communal disposal 740 (e.g., trash, or the like).

FIG. 7B illustrates an enlarged view of a ghost station 705 present on the second floor (or other floor) of the multi-vendor drive-through complex, in accordance with embodiments of the invention. As shown, each of the one or more ghost stations 705 may comprise one or more modules associated with preparation of food. Each of the one or more modules may be configured based on the requirements of the vendor occupying a ghost station 705. For example, a dessert serving ghost station 705 may comprise one or more refrigeration modules. In another example, a fast food serving ghost station 705 may comprise one or more fryers. In an exemplary embodiment, a ghost station 705 may comprise one or more freezers 770, an assembly station 750, and cooking station 760. In some embodiments, the ghost station 705 may comprise one or more dumbwaiters 624 and/or one or more robotic loading stations for providing products to one or more robotic delivery systems 1100 that may move between vendors to collect sub-orders, as will be described in further detail herein. The placement of the one or more modules shown in FIG. 7B is for illustrative purposes only and may be configured based on the requirements of the one or more vendors.

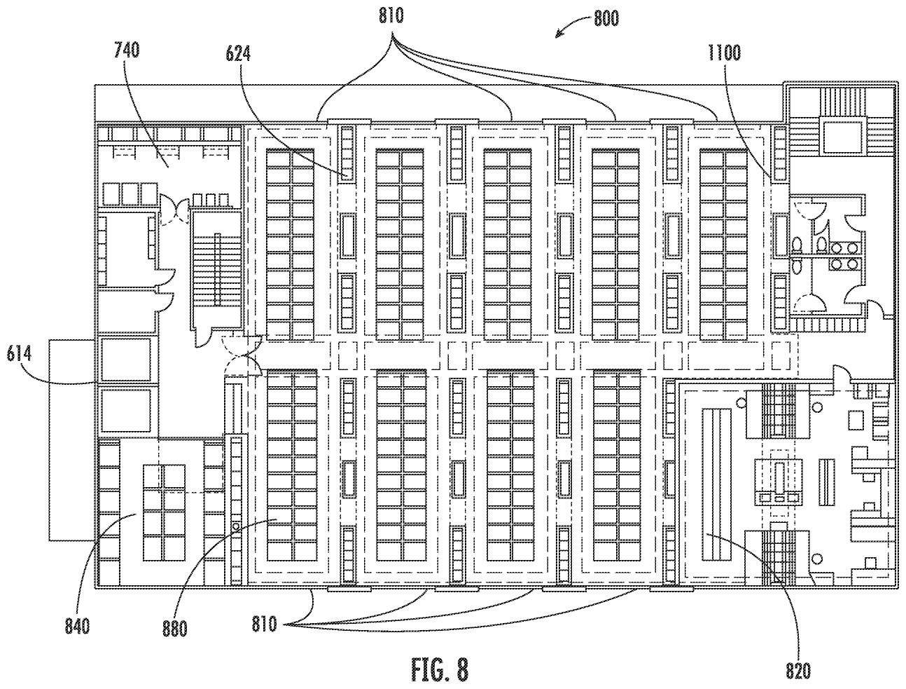

FIG. 8 provides a site plan for the third floor 800 (or other floor) of the multi-vendor drive-through complex, in accordance with an embodiment of the invention. The third floor 800 may be configured for a pharmacy, an e-commerce warehouse, a hardware store, a clothing store, or the like vendor described herein or not specifically described herein. For explanatory purposes, third floor 800 is considered to be occupied by a pharmacy. As shown, the site plan for third floor 800 may comprise one or more storage bays 810, where each of the storage bays 810 comprises one or more storage bins 880 for storing any type of good, such as but not limited to food (e.g., packaged food, drinks, or the like), personal items (e.g., toiletries), healthcare items (e.g., make-up, or the like), gifts, over the counter medicines, or the like. In some embodiments, one or more of the storage bins 880 may be refrigerated bins that have specific temperature controls required for storing different types of medicines. The site plan for the third floor 800 may comprise a pharmacy area 820 to store prescription medicines and/or equipped with one or more computing devices that allows one or more pharmacists to communicate with one or more users in the drive-through lanes via the one or more pick-up stations 622 (e.g., video interfaces within or adjacent to the kiosks) to answer questions, provide instructions about the medicines being picked up, or the like. The site plan for the third floor may further comprise a storage space 840 and a personnel locker facility, communal disposal 740, or the like. In some embodiments, each of the storage bays 810 may further comprise one or more vertical transport systems (e.g., dumbwaiters 624, elevators, or the like), one or more horizontal transport systems (e.g., conveyers, or the like), one or more material handling robots (e.g., robots with moveable arms, grips, or the like), and/or robotic loading stations for providing products to the one or more robotic delivery systems 1100 (e.g., designated areas for loading the sub-orders/orders to the robotic delivery systems 1100).

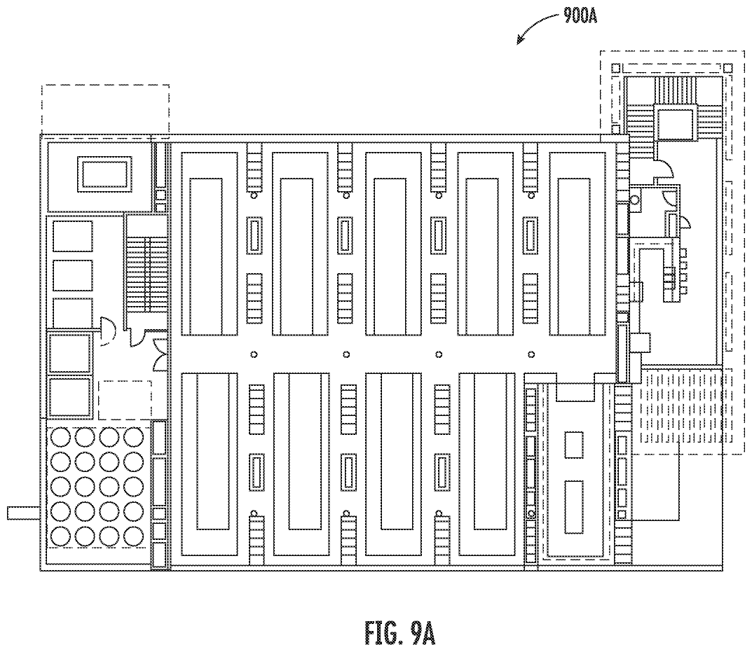

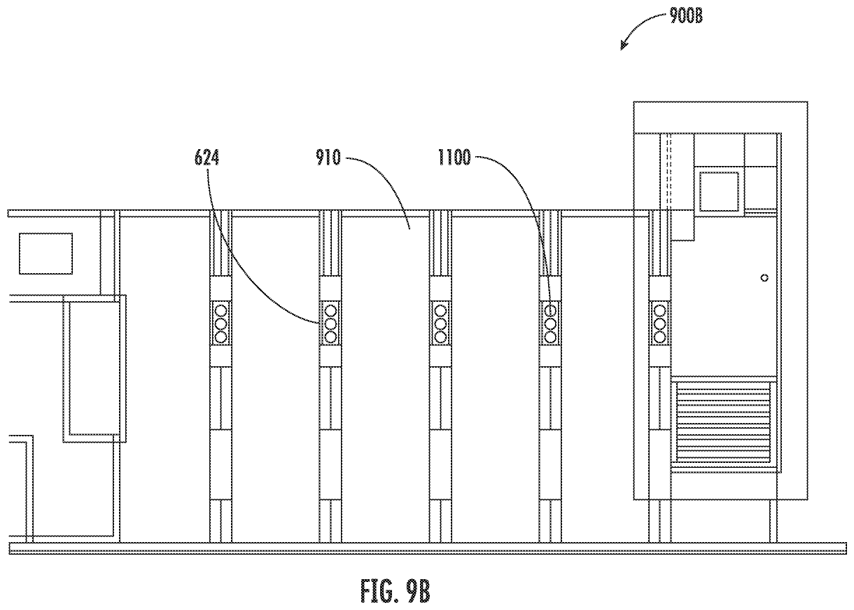

FIG. 9A provides a site plan for the fourth floor 900A (or other floor) of the multi-vendor drive-through complex, in accordance with embodiments of the invention. As shown, the fourth floor may be a mezzanine level and/or a roof comprising storage areas, HVAC units, maintenance areas, drone pick-up areas, or the like. FIG. 9B provides a site plan for the roof 900B of the multi-vendor drive-through complex, in accordance with embodiments of the invention. As shown, the site plan for the roof may comprise one or more drone pick-up areas 910 that allow one or more drones to pick up one or more orders and deliver it to the one or more users after receiving the orders via one or more vertical transport systems (e.g., dumbwaiters 624, elevators, or the like) and/or one or more robotic delivery systems 1100. In some embodiments, the done pick-up area 910 may include pick-up stations 622 that secure orders until the drones pick-up the orders of the pick-up stations 622.

Moreover, in some embodiments of the invention, the floors, the mezzanine, the roof, and/or the like may include pick-up stations 622. For example, should the complex have offices, a common area (e.g., private or public), a public access area, or the like, the pick-up stations 622 may be located throughout the complex 600. In particular, should the ground level, mezzanine, roof, or other intermediate floors of the complex 600 have a common area (e.g., public cafeteria, or the like) users (e.g., employees, public users, or the like) may place an order and have it delivered to a pick-up station 622 within the complex (e.g., common area) by the robotic delivery systems 1100 using the vertical transport systems (e.g., that connect the floors, including the ground floor, intermediate floors, roof, or the like) so that the user my pick-up the order, and either leave the site or dine within the complex (e.g., the common area, an office, or the like).

As illustrated in the figures, the vertical transport systems (e.g., dumbwaiters 624, elevators, or the like) interconnect each of the floors and allow for a single order from a vendor to be delivered to a pick-up station 622 on the ground floor. Alternatively, multiple orders from different vendors on the same floor and/or different floors may be combined using the horizontal transport systems (e.g., conveyers, or the like), vertical transport systems (e.g., dumbwaiters 624, elevators, or the like), and/or robotic delivery systems 1100 within each of the floors which allows for goods from different areas of a floor and/or from different floors to be combined and delivered to a pick-up station 622. In some embodiments, the vertical transport systems 1100 may comprise a UV light feature for purposes of performing cleaning.

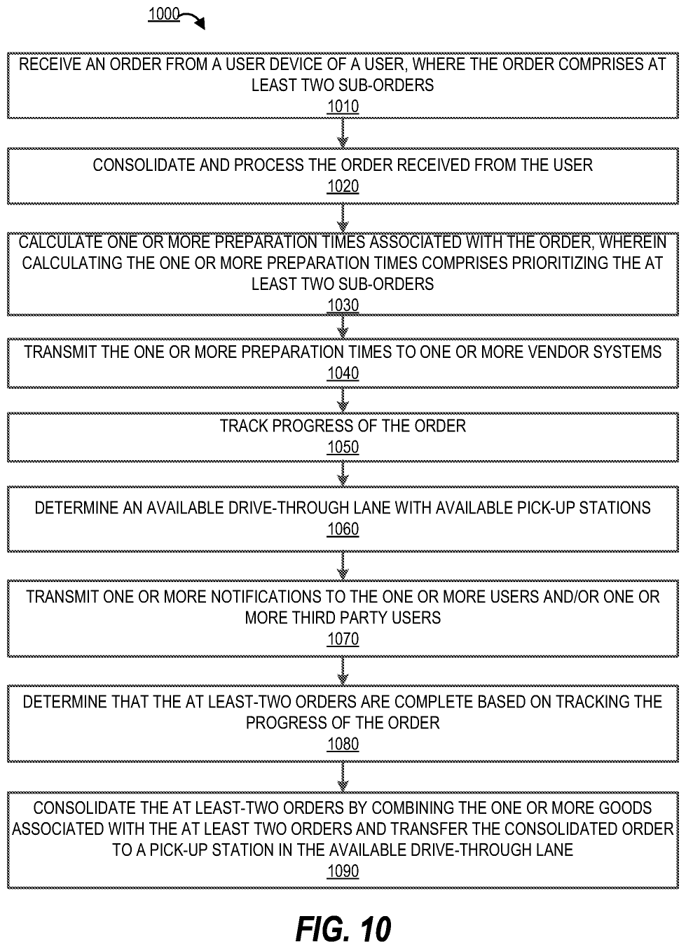

FIG. 10 provides a flowchart illustrating a process flow for facilitating order placement, consolidation, and fulfillment of multi-vendor orders, in accordance with embodiments of the invention.

As shown in block 1010, the present invention receives an order from a user device of a user (e.g., customer user, third-party user, or the like), where the order comprises at least two sub-orders. In some embodiments, the at least two sub-orders may be associated with goods belonging to the same category (e.g., food). In some embodiments, the at least two sub-orders may be associated with goods belonging to different categories (e.g., food, medicines, hardware, groceries, or the like).

As shown in block 1020, the present invention consolidates and processes the order received from the user. The system may consolidate and process the order by communicating with one or more vendor systems 200. Consolidating and processing the order may comprise at least checking the inventory and verifying that the order can be fulfilled by the one or more vendor systems 200. In some embodiments, an employee input may be required to process the order, where the employee may provide the input via the one or more vendor systems 200.

As shown in block 1030, the present invention calculates one or more preparation times associated with the order. Calculation of the one or more preparation times may comprise prioritization of the at least two sub-orders associated with the order and the prioritization may be based on location of user, real-time order backlog associated with the one or more vendors, type of each of the sub-orders, preparation time associated with each of the sub-orders, pick-up time set by the user, and/or the like. For example, the order may comprise a first sub-order associated with fast food, a second sub-order associated with a dessert, and a third sub-order associated with medicines, the system determines the current order backlog associated with the fast food ghost station, the dessert ghost station, and pharmacy and calculates that the preparation of the third sub-order should be started at X time, the preparation of the first sub-order should be started at Y time, and the preparation of the second sub-order should be started at Z time, where ‘Z’ may be greater than ‘X’ and ‘Y’ and ‘Y’ may be greater than ‘X.’ In other words, the system prioritizes the preparation times of the one or more sub-orders based on the factors mentioned above. As shown in block 1040, the present invention transmits the one or more preparation times to one or more vendor systems 200.

As shown in block 1050, the present invention tracks progress of the order. The system tracks the progress of the order by communicating with the one or more vendor systems 200. As shown in block 1060, the present invention determines available robotic delivery systems 110 and/or available drive-through lane with available pick-up stations 622 based on one or more factors, where the one or more factors comprise type of the order, storage requirements for order (e.g., hot/cold/room-temp/ambient temp), size of the order, estimated completion time, estimated pick-up time of the user, and/or the like. In some embodiments, the system may also determine an available robotic delivery system 1100 and/or pick-up station 622 based on the type of the order. Continuing with the previous example, the system may select a robotic delivery system 1100 and/or pick-up station 622 with hot, cold, and/or other types of storage compartments. In some embodiments, the system may determine an available robotic delivery system 1100 and/or pick-up station 622 based on size of the order. For example, a larger robotic delivery system 1100 (e.g., with a larger compartment(s)) and/or a larger pick-up station 622 may be required for a large order, while a smaller a robotic delivery system 1110 (e.g., with smaller compartment(s)) and/or a smaller pick-up station 622 may be available for a single product from a single vendor. In some embodiments, the system may determine the available robotic delivery system 1100 and/or pick-up station 622 based on the estimated completion time of the order, estimated pick-up time of the user, and/or the like.

As shown in block 1070, the present invention transmits one or more notifications to the one or more users and/or one or more third party users. The one or more notifications may be associated with the progress of the order, the drive-through lane information, pick-up stations, or the like determined in block 1060, or other like notification. It should be understood that the notification may be sent to the user computing device system 400 before or after the user computing device system 400 is detected on-site. When the user arrives at the designated pick-up station 622, the user may be able to access the user order in any number of ways, such as using an identifier (e.g., using a code sent in the notification, using a feature on the computing device 400 to communicate with the pick-up stations 622, automatically based on the system identifying the presence of the computer device 400 at the pick-up station 622, or the like). The user may check the order, and should the order be incorrect the user may notify the one or more vendors through the user's computing device 400 (e.g., call, action through an application, or the like). The user may then wait in a designated parking spot for manual delivery of a corrected order. Alternatively, the user may be directed back to a pick-up station 622 when the order is corrected.

Should a user arrive on-site before the order is completed the user may be directed to a parking spot until being notified that the order is complete, at which time the user may be directed to a drive-through lane and pick-up station 622 to collect the order.

It should be further understood that sensors (e.g., cameras, infrared, LiDAR, or the like) may be incorporated into the lanes (e.g., drive-through lanes, autonomous vehicle lines, robotic delivery lanes), robotic delivery systems 1100, and/or pick-up stations 622 in order to determine the presence of a vehicle, type of vehicle being used by the user, perform facial recognition while validating at least one of the sub-orders associated with an order (e.g., validating prescription by the pharmacy). In response, the pick-up station 622 may be adjusted (e.g., changed to a different kiosk, the kiosk may be lifted, a shelf in the kiosk may be raised or lowered, or the like) in order to accommodate the height of the vehicle of the user picking up the order.

As shown in block 1080, the present invention determines that the at least-two sub-orders are complete based on tracking the progress of the order. As shown in block 1090, the present invention consolidates the at least-two orders by combining the one or more goods associated with the at least two sub-orders and transfer the consolidated order to a robotic delivery system 1100 and/or a pick-up station 622 in the available lane. For example, the order may comprise a first sub-order associated with fast food, a second sub-order associated with a dessert, and a third sub-order associated with medicines. Upon determining that the first sub-order, the second sub-order and the third sub-order are complete based on tracking the progress of the order, the employees, horizonal transport systems, handling robots, and/or robotic delivery systems 1100, or the like may consolidate the first sub-order, the second sub-order and the third sub-order and place the consolidated order on a the robotic delivery systems 1110, vertical transport systems, and/or the like, where the consolidated order is transferred to the pick-up station determined in step 1060 (e.g., through the robotic delivery systems 110, dumbwaiters 624, or the like). In some embodiments, parts of the process described herein may require input (partial input and/or complete input) from personnel associated with the multi-vendor drive-through complex and/or the one or more vendor systems 200.

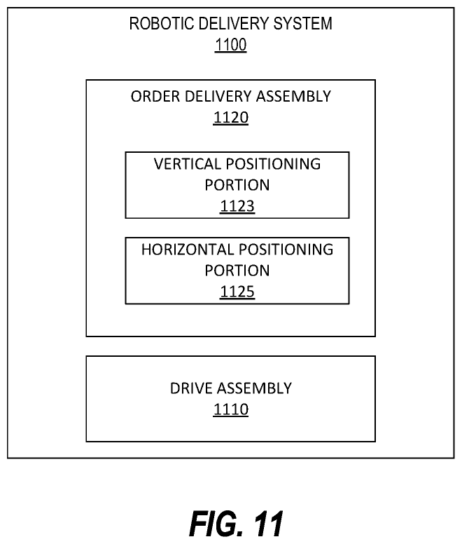

FIG. 11 provides a block diagram illustrating embodiments of a robotic delivery system 1100 for delivering multi-vendor orders from one or more floors to one or more pick-up stations 622 (or for retrieving expired orders that have not been picked up from the pick-up stations 622). As shown, the robotic delivery system 1100 comprises at least an order delivery assembly 1120 to receive an order from multi-vendors on one or more floors and to deliver the at least one order to the one or more pickup stations 622, and a drive assembly 1110 configured to move the robotic delivery system 1100, such as horizontally, vertically, rotationally, or the like such that the robotic delivery system 1100 may move in any direction. In some embodiments, the drive assembly 1110 is at least partially located below the order delivery assembly 1120. The drive assembly 1110 may comprise one or more wheels (e.g., one or more driven wheels, one or more free wheels, or the like), one or more tracks, one or more legs, or the like operatively coupled to one or more motors (e.g., to an electric motor, or the like), one or more drive trains, one or more actuators, one or more gears, or the like that allows for the movement of the robotic delivery system 1100 in any direction.

In some embodiments, the one or more robotic delivery systems 1100 may be confined to specific areas on each floor. In some embodiments, the one or more robotic delivery systems 1100 may be allowed to access the entire floor. As such, the one or more robotic delivery systems 1100 may move between stations at multiple vendors, to bays where products are stored, or the like to receive orders from multiple vendors. Additionally, or alternatively, the one or more robotic delivery systems 1100 may collect orders from one or more consolidation areas 715, where products from multiple or single vendors have been assembled. In some embodiments, the one or more robotic delivery systems 1100 may move between the one or more floors to the robotic delivery lanes and/or the one or more drive through lanes through the vertical transport system, such as an elevator sized for one or more of the robots. As previously described herein, the one or more robotic delivery systems 1100 may move to the one or more pick-up stations 622 through robot delivery lane.

The orders within the robotic delivery system 1100 may be received and/or delivered through the use of an order delivery assembly 1120. The order delivery assembly 1120 may comprise one or more containers (e.g., vessel, tray, cubby, tote, bag, or the like), one or more clips, tracks, carriages, slides, actuators, or the like that may receive orders, position the orders for delivery, and/or deliver the orders. As such, the order delivery assembly 1120 may comprise a vertical positioning portion 1123 for receiving an order at a particular height and/or for moving the order vertically within the robotic delivery system 1100 to change the vertical location of an order for delivery to the one or more pick-up stations 622. For example, the vertical positioning portion 1123 may move a container within the robotic delivery system 1100 to the desired height to receive one or more products for an order from one or more vendors. Moreover, during and/or after the robotic delivery system 1100 is moving around a floor, down a vertical transport system, and/or to the pick-up station 622, the vertical positioning portion 1123 may move the order (e.g., in a container, or the like) to position the order for delivery. Moreover, in some embodiments, the order delivery assembly 1120 may comprise a horizontal positioning portion 1125 for moving the order horizontally from the robotic delivery system 1100 to the one or more pick-up stations 622. For example, the horizontal positioning portion 1123 may move the container, the order on the container, or the like such that the order is delivered from the robotic delivery system 1100 to the pick-up station 622.

It should be further understood that in some embodiments, the robotic delivery system 1100 may include temperature controlled sections (e.g., the containers, compartments, or the like in which the orders are stored) that keep orders (e.g., food, prescriptions, or the like) hot, cold, room-temp, ambient-temp, or the like. However, since the orders are processed in real-time and/or delivered to the pick-up stations 622 as previously described herein, the robotic delivery systems 1110 may not require temperature controlled sections.

The robotic delivery systems 1110 that pick-up orders from one or more vendors and deliver the orders, such as to the pick-up stations 622, drone delivery area 910, and/or other location may be moving the majority of the time (e.g., constantly when operating) in order to receive and deliver orders. However, in some embodiments the one or more robotic delivery systems 1110 may return to a charging station when the robotic delivery systems 1110 have low power, when the complex is closed, when the complex is in non-peak operation (e.g., late at night, between peak breakfast, lunch, and dinner times), or the like. In some embodiments, the robotic delivery systems 1110 may be charging when they are stationary at specific locations, such as when receiving orders from vendors, when traveling up and down the vertical transport systems (e.g., dumbwaiter, elevators, or the like), when delivering orders to the pick-ups stations 622, or the like. As such, at particular locations within the complex the robotic delivery systems 1110 may be at least partially charged as they are operating and/or the complex may have dedicated charging locations at which the robotic delivery systems may charge as needed. The charging of the robotic delivery systems 1110 during operation and/or during downtime will aid in making the robotic delivery systems 1110 available during peak operating times.

In should be further understood that while the robotic delivery systems 1100 are generally discussed as one type of system, different types of robotic delivery systems 1100 may be used for different tasks. For example, some robotic delivery systems 1100 may be used strictly for picking products (e.g., different types of robots may be used to pick orders from the ghost kitchen, versus products from a pharmacy, or the like), some may be used for consolidating orders, some may be used for delivering orders to user vehicles and/or the pick-up stations 622, some may be used for delivering orders to autonomous delivery vehicles, some may be used for delivering orders to bike, scooter, walk-up stations, or the like. As such, different combinations of robotic delivery systems 1100 may be used for different tasks within the complex. As such, the robotic delivery systems 1100 (e.g., single type of system, or multiple types of systems) may move between multiple vendors on a single floor and/or between floors to pick one or more orders from different locations (e.g., a single order or multiple orders from vendors). Additionally, or alternatively, the robotic delivery system 1100 may consolidate orders and deliver the orders to a consolidation stations 715, and other robotic delivery systems 1100 may take the consolidated orders from the consolidation stations 715 to the pick-up stations 622.

It should be understood that the robotic delivery systems 1100 may have components that are the same as or similar to the other systems described herein. For example, one or more processing devices, one or more network communication interfaces, one or more memory devices, or the like that allow the robotic delivery systems 1100 to communicate with the other system and applications described herein.

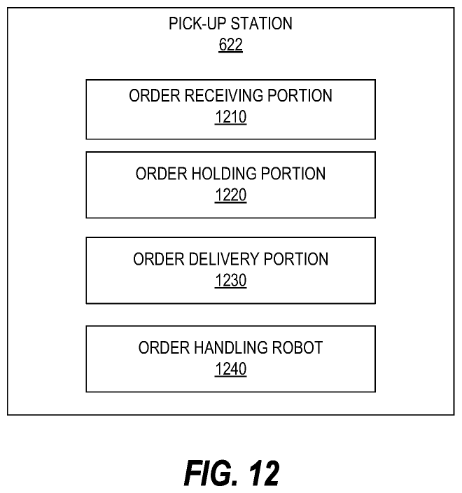

FIG. 12 provides a block diagram illustrating embodiments of the pickup-station 622 for receiving, storing, and delivering multi-vendor orders, in accordance with embodiments of the invention, and in particular, for receiving orders from a robotic delivery system 1100. In some embodiments, the one or more pick-up stations 622 may comprise one or more of an order receiving portion 1210, an order holding portion 1220, an order delivery portion 1230, and/or an order delivery robot 1240. The order receiving portion 1210 may be configured to receive the order from the one or more robotic delivery systems 1100. As such, the order receiving portion 1210 may be located on the backside of the pick-up station 622. The order receiving portion 1210 may include a door (e.g., swinging, sliding, retracting, or the like) that may be opened when the robotic delivery system 1110 is adjacent the pick-up station 622 (e.g., approaches the pick-up station, is located next to the pick-up station, or the like). Alternatively, the order receiving portion 1210 may be open on the backside of the pick-up station. The order holding portion 1220 may be configured to hold the order until the user and/or autonomous delivery vehicle is identified as present. The order holding portion 1220 may be adjustable vertically and/or horizontally to aid in receiving the order from the robotic delivery system 1100. Moreover, the order holding portion 1220 may be temperature controlled (e.g., heated, cooled, room-temp, ambient-temp, and/or the like) depending on the type of order. The order delivery portion 1230 may be configured to deliver an order to the user once the user is identified as being located adjacent the pick-up station 622, as previously described herein. The order delivery portion 1230, like the order receiving portion 1210, may include a door that opens when the user is identified as being at the pick-up station 622. In some embodiments, the order delivery portion 1230 may extend the container containing the order and/or extend the order itself to the vehicle (e.g., to a user in the vehicle, to a compartment within an autonomous delivery vehicle, or the like). It should be understood that each pick-up stations 622 may include one or more order receiving portions 1210 (e.g., a single door or multiple doors for receiving an order), one or more order holding portions 1220 (e.g., a single compartment or multiple compartments), and/or one or more order delivery portions 1230 (e.g., a single door or multiple door for allowing a user to access an order). As such, a pick-up station 622 may include a single compartment (e.g., a single locker section, kiosk section, or the like) or multiple compartments (e.g., multiple locker sections, kiosk sections, or the like stacked on top of and/or next to each other to form a bank of compartments).

In some embodiments the pick-up station 622 may include, or have located adjacent to the pick-up station 622, or the robotic delivery system 1110 may include, an order handling robot 1240 that is configured to pick an order from the one or more robotic delivery systems 1100 and deliver the order to the order holding portion 1220 of the pick-up station 622. For example, the order handling robot 1240 may include one or more arms and a grip that is used to pick orders from the robotic delivery systems 1100 and deliver it to the order holding portion 1220 of the pick-up station 622.

Consequently, it should be understood, and as previously described herein, the pick-up stations 622 (e.g., kiosks, lockers, or the like) may be stand-alone stations in which a single order may be delivered or a bank of stations in which multiple orders may be provided until the users pick-up the orders.

It should be understood that the pick-up stations 622 may have components that are the same as or similar to the other systems described herein. For example, one or more processing devices, one or more network communication interfaces, one or more memory devices, or the like that allow the pick-up stations 622 to communicate with the other system and applications described herein.

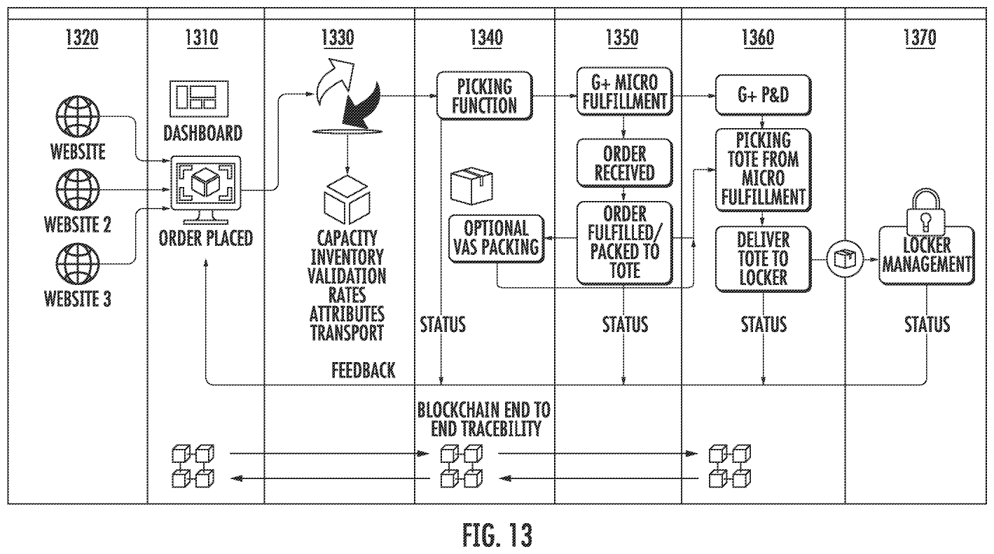

FIG. 13 further illustrates a process flow under which orders are made, processed, and delivered to the pick-up stations 622. As illustrated by blocks 1310 and 1320 in FIG. 13, an order may be placed through an interface (e.g., user computer system, interface at the complex, or the like), in which a user may select products for an order from multiple vendors directly from a centralized dashboard, or indirectly by accessing websites (or portions of websites) of the vendors that are displayed on the interface.

As illustrated by block 1330 in FIG. 13, and as previously discussed herein with respect to the multi-vendor order fulfillment systems 300 and/or the other systems, the systems determine the procurement of the products for orders, such as but not limited to the availability (e.g., location, inventory, time to make, or the like) of the products, the capacity for the vendors to provide the product (e.g., based on other orders in the queue for the vendors, or the like), the availability of the robotic delivery systems 1100 (e.g., locations, orders being delivered, queue of orders in line, or the like for the robotic delivery systems), the time to pick and transport the products, when the user would like to pick-up or have an order delivered, or the like.

FIG. 13 further illustrates in block 1340 that the picking and/or packing of the products for the orders begins (e.g., the portion of the order that may have the longest throughput, or the like), and in block 1350 illustrates that the picking and/or packing is completed by consolidating the order in the container (e.g., tote, tray, bag, or the like as previously discussed herein). FIG. 13 further illustrates that the container may be picked and/or packaged in a consolidation area and/or may be consolidated as the order is being fulfilled. Regardless, the container may be delivered to the pick-up stations 622 (e.g., kiosk, locker, or the like), as illustrated by block 1360 of FIG. 13.

FIG. 13 further illustrates in block 1370 that the order is placed in the pick-up station 622 (e.g., in the locker) where it can be opened by the user once the user arrives on site and/or at the pick-up station 622. As further illustrated in FIG. 13, notifications (e.g., order status, or the like) of the order and/or the products thereof may be sent to the vendors within the complex in order to keep the orders on-time, note a delay in an order, note an update to an order, or the like. Moreover, in some embodiments a user may be able to receive a notification of the status of the order (e.g., time until an order is ready, or the like). Moreover, the when the order is in the process of being completed, complete, being delivered to a pick-up station 622, or the like, the user is notified of the location of the pick-up stations 622 to which the order is or will be delivered (e.g., lane, station within the lane, and/or station within a bank of stations within the lane, or the like) and/or provided instructions for accessing the order through the pick-up station 622. As previously discussed herein, the drive-through system 203, the multi-vendor order fulfillment system 300, and/or other systems in the complex control the instructions provided to a user or automatous delivery vehicle in order to direct the vehicle (e.g., user or automatous vehicle), before or as the vehicle arrives, to the order in the correct lane and/or the pick-up stations 622; controls the delivery of the order from the pick-up station 622 to the vehicle; allows the user to access the correct order from the pick-up station 622 when the user arrives at the correct pick-up station 622 using an identifier (e.g., after opening a locker using a code, communication between the user computer system and the locker, after the user is verified by the systems, or the like); and/or directs the vehicle out of the complex after the order is received.

In some embodiments, it should be understood that the ordering, fulfillment, and/or delivery of the order, as previously discussed with respect to FIG. 13, may be tracked and/or controlled by blockchain technology. For example, the blockchain technology may receive, process, and/or store the orders for accuracy, efficiency, and verification.

FIGS. 14 through 16 illustrate site plans for the ground floor of the multi-vendor drive-through complex 600, in accordance with embodiments of the invention. In some embodiments, the drive through complex 600 may provide improved traffic flow 1400 through the complex. For example, as described generally, the property of the complex may have multiple ingress 1410 and egress 1412 locations for vehicles (e.g., any vehicle, or for specific vehicles in different locations) to enter and exit the property. In some embodiments, autonomous delivery vehicles may have a specific location for ingress and egress to and from the property (e.g., to separate the autonomous delivery vehicles from user vehicles). The property may further comprise a staging area 1430 before orders are received by the users in the vehicles, and an access area 1420 (e.g., departing area, return lanes, or the like) for vehicles leaving after orders are received and/or moving around the property (e.g., for accessing parking, returning to the staging area 1430 for order correction, or the like). In some embodiments, the staging area 1430 may comprise staging lanes 1432 at which the flow of traffic may be controlled to each drive through lane 601. For example, the vehicles may be identified as they enter the property, move through the property, and are waiting in the staging area 1430. The staging area 1430 may include staging lanes 1432 and/or staging traffic control elements 1434 (e.g., lights, electric signs, traditional signs, way-finding signage, or the like) that may label different staging lanes 1432 and/or direct the user vehicles to the drive through lanes 601 (e.g., specific lanes based on the user and/or vehicle identified). The access area 1420 may allow for improved traffic flow out of the drive-through lanes 601. In the illustrated embodiment, the access area 1420 may communicate with the drive-through lanes 601. In the illustrated embodiment, the access area 1420 comprises two lanes 1422 for opposing traffic that allows for vehicles leaving the drive-through lanes 601 to exit the property more easily through the egresses 1412 of the property. As such, the access area 1420 may also comprise of exit traffic control elements 1424 (e.g., lights, electric signs, traditional signs, way-finding signage, or the like). As such, the complex 600 may control the timing of when users (e.g., user vehicles, bicycles, scooters, pedestrians, or the like lanes) and/or autonomous delivery vehicles enter and/or leave the complex 600. For example, as users and/or autonomous delivery vehicles enter the property of the complex 600, the drive-through systems 203 may hold the vehicles in lane staging areas 1430 before the vehicles are directed to particular lanes 601 and/or pick-up stations 622. The flow of vehicles can be sped up or slowed down in the staging areas 1430, in the lanes 601, and/or in departing exit lanes to control the flow of vehicles entering, located within, and/or leaving the property. The movement of the vehicles may be based at least in part based on information received from surrounding traffic systems (e.g., lights, cameras, sensors, or the like) to determine when traffic is clear/reduced and/or heavy/backed-up on the surrounding streets around the property.

FIGS. 14 through 16 will now be described in further detail in which the staging areas 1430 are described as having different zones that allow for the improved traffic flow through the property. For example, the zones may include one or more feeding zones 1450, 1470 that are used to direct vehicles for which the orders are ready to the merging zone 1480 that merge and direct the vehicles to the pick-up zone 1490 where the vehicles pick-up orders from the pick-up stations 622. Moreover, the zones may include one or more holding zones 1460 for vehicles that arrive early, are picking up orders that have expired (e.g., have been removed from the pick-up station 622 by the automatic delivery systems 1100, or the like), and/or have not yet placed an order. As such, as previously described herein, the drive-through system 203, traffic control elements 1424, user computer systems 400 (e.g., GPS, other location determination devices), autonomous vehicle systems, other systems described herein, or the like, may communicate with each other in order to direct the flow of traffic through the property. As previously described herein, global positioning systems (GPS) or other location determination device (e.g., near-field communication, mesh networking, wi-fi triangulation, or the like sensors) may be used to identify the location of the vehicles with respect to the zones (e.g., sensors at the zones, lanes, individual parking spaces, or the like) and/or pick-up stations 622 (e.g., sensors thereof) within the property in order to direct the flow of traffic based on the status of the order, locations of the vehicles arriving at the property, and/or located on the property (e.g., in the zones, lanes, and/or spaces), or the like.

FIGS. 14 through 16 illustrate that one or more feeding zones 1450, 1470 may be used to direct vehicles that are on-time (e.g., within ½, 1, 2, 3, 4, 5, 6, 7, 8, 9, 10, or the like minutes from the property) and/or late for picking up orders (e.g., arriving and/or arrived after the order has been delivered to the pick-up station 622). For example, a first feeding zone 1452 (e.g., queuing lanes, or the like) may be used for vehicles that may be on-time (e.g., within seconds, minutes, or the like of pick-up time, or the like) and/or late (e.g., within seconds, minutes, or the like after an order has been delivered to a pick-up station) for picking up orders. As such, when vehicles are on-time and/or late arriving to the property they may be directed to the first feeding zone 1452. The first feeding zone 1452 may be used to feed vehicles to a second feeding zone 1454 (e.g., pole-position lines) in which the vehicles are positioned properly with respect to the drive-through lane 601 having the pick-up station 622 in which the order for the vehicle is located. In some embodiments, the feeding zone 1450 (e.g., first zone 1452, second zone 1454, or the like) are moving consistently because the orders for these vehicles have been delivered and/or or about to be delivered to the pick-up station 622. As such, it should be understood that in some embodiments when vehicles are identified as being a particular distance and/or away from the property (e.g., within 1, 2, 3, 4, or the like minutes) a command may be sent for loading the pick-up station 622 (e.g., a 20, 30, 40, 50, 60, or the like second warning) with the order to provide enough time for the orders to be delivered to the pick-up stations 622.

FIGS. 14 through 16 further illustrate that one or more holding zones 1460 may be used to direct vehicles that are early (e.g., more than 2, 3, 4, 5, 6, 7, 8, 9, 10, or the like minutes early), vehicles that have arrived after an order has expired (e.g., late that the order has been removed from the pick-up station 622), and/or vehicles that have arrived on site with a non-order (e.g., before placing and order). For example, a third zone 1462 (e.g., or a first holding zone) may be used for early arrivals and/or a fourth zone 1464 (e.g., or a second holding zone) may be used for expired orders and/or pre-orders. However, it should be understood that the third zone 1462 and/or the fourth zone 1464 may be a single zone. In some embodiments, when the orders for the early arrivals, reprocessed expired orders, and/or on-site orders are ready, the orders may be brought to the holding zone 1460, or the vehicles in the holding zones 1460 may be directed to proceed to other zones, such as the one or more feeding zones 1450 and/or one or more secondary feeding zones 1470. The holding zone 1460 may include other features such as vehicle charging stations that may be used to charge the vehicles while users wait, terminals (e.g., that users may use to place an order if the user does not have a user computer system, or the like), information regarding the usage and products of the complex, or the like.

As further illustrated in FIGS. 14 through 16, one or more auxiliary feeding zones 1470 may include a fifth zone 1472 (e.g., a first auxiliary feeding zone 1472 that includes auxiliary pole-position lines, or the like) in which the vehicles that were located in the one or more holding zones 1460 are directed and/or positioned properly with respect to the drive-through lanes 601 once the order for the vehicles in the holding zone 1460 are ready for pick-up.

Consequently, the one or more feeding zones 1450, 1470 (e.g., primary and/or auxiliary feeding zones) are used to direct the vehicles (e.g., counter-clockwise and clockwise, respectively) to the proper drive-through lanes 601 through one or more merging zones 1480 (e.g., a sixth zone, a first merging zone 1482, or the like). The merging zone 1480 allows opposing traffic from the one or more feeding zones 1450, 1470 to feed into the pick-up zone 1490 (e.g., a seventh zone, or a first pick-up zone) that includes the drive-through lanes 601, as previously discussed herein. Traffic control elements 1424 (e.g., overhead lights, overhead signs, ground signs, ground lights—such as lane lights, or the like) may be utilized along with the drive-through systems 203 to control the flow of traffic from the one or more feeding zones 1450, 1470, through the merging zone 1480, and/or through the pick-up zone 1490. In the illustrated embodiments in FIGS. 14 through 16, the pick-up zone 1490 has four drive-through lanes 601, each having four pick-up stations 622, while each pick-up station 622 has a bank of four compartments (e.g., four separate lockers, or the like). While FIGS. 14 through 16 illustrate a particular number of drive-through lanes 601 and/or pick-up stations 622, it should be understood that any number of drive-through lanes 601 and/or pick-up stations 622 (including the any number of one or more compartments within each pick-up station 622) may be utilized. As previously described herein, as the vehicle approaches the pick-up station 622 and/or reaches the pick-up station 622, authentication of the user occurs (e.g., the user presents credentials) in order to pick-up orders from the pick-up stations 622.

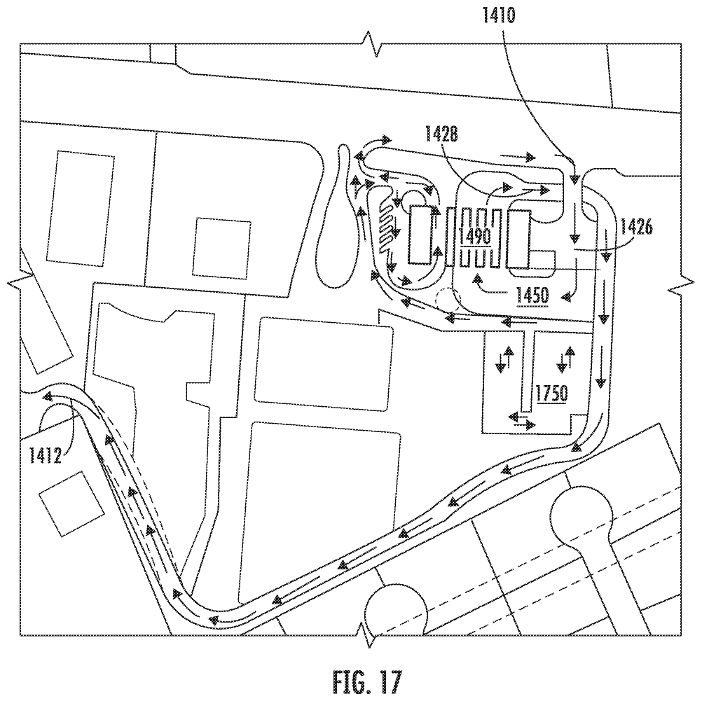

While specific zones are described and illustrated herein with respect to FIGS. 14 through 16, and other portions of this application, it should be understood that any number of zones may be used and/or located in different parts of the property in order to accomplish the features and functionality described herein. Moreover, FIG. 17 provides additional embodiments of the complex 600 that improves traffic flow within the multi-vendor complex 600. As illustrated in FIG. 17, in some embodiments the complex may have one or more ingresses 1410 and one or more egresses 1412 for vehicles in different locations and/or and access areas 1420 that overlap each other. For example, one or more entry access areas 1426 may lead vehicles to the one or more feeding zones 1450, 1470 (e.g., primary and/or auxiliary feeding zones) or directly to the pick-up zone 1490 (e.g., the one or more drive-through lanes 601) and/or the medical services section 1500 (described in further detail below). One or more exit access areas 1428 may lead vehicles from the pick-up zone 1490 (e.g., the one or more drive-through lanes 601) and/or the medical services section 1500 (described in further detail below) to one or more egresses 1412. In some embodiments, the inlet access area 1426 and the exit access area 1428 may at least partially overlap. As illustrated in FIG. 17, in some embodiment the exit access area 1428 may extend below the inlet access area 1426; however, it should be understood that in some embodiments the exit access area 1428 may extend over the inlet access area 1426. As such, the premises of the complex 600 may include tunnels, bridges, or the like, such that at least a portion of one or more exit access areas 1428 and the one or more inlet access areas 1426 do not cross (e.g., extend over, under, or the like each other) to improve upon the flow of vehicles in and out of the complex 600, and in particular, in and out of the pick-up zone 1490 and/or the medical services sections 1500.

As previously discussed herein, the ground floor of the multi-vendor drive-through complex 600 may comprise one or more drive-through lanes 601 (e.g., 602, 604, 606, 608, 610) for user vehicles, one or more autonomous vehicle lanes for autonomous vehicles, one or more robotic delivery lanes for the robotic delivery systems 1100, or the like. However, FIGS. 14 through 16 further illustrate that the ground floor may also include a medical services section 1500, in which medical services may be provided to users either while the users remain in their vehicles or while users park and enter exam rooms.

As illustrated in FIG. 14 the medical services section 1500 may include one or more drive through lanes 1502 (e.g., a center lane that is used to enter and exit the medical services section 1500) and/or one or more parking lanes 1504 with one or more medical parking locations, such as in front of medical services stations 1510. The medical services stations 1510 may serve as exam rooms, supply rooms, and/or offices for medical professionals (e.g., doctors, nurses, practitioners, or the like). The medical services stations 1510 may operate and/or provide the medical services that are typically provided within drug stores, superstores, pharmacies, urgent care facilities, or the like, such as shots, tests (e.g., flu, corona viruses, other viruses, x-rays, scans, or the like), diagnoses, minor medical procedures (e.g., stiches, or the like), casts, eye care services (e.g., eye exams, glasses, contracts, or the like).

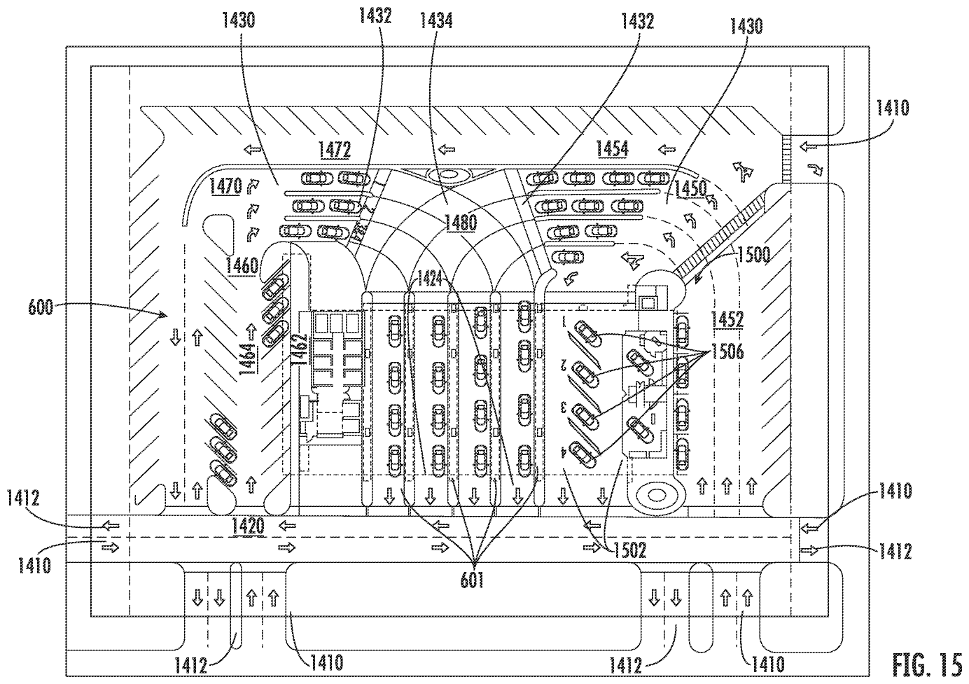

In alternate configurations, as illustrated in FIG. 15, the medical services section 1500 may have one or more drive through lanes 1502 and/or one or more medical parking stations 1506. For example, the one or more medical parking stations 1506 may be one or more first medical parking stations 1508 and one or more second medical parking stations 1509 (e.g., medical bays, or the like). The one or more medical parking stations 1506 may operate as the medical service stations 1510. In some embodiments, the one or more first medical parking stations 1508 may be open air bays in which users may remain in their vehicles and receive medical services. As such, the open air bays may include medical service devices (e.g., diagnostic equipment, thermometers, blood pressure monitors, heart rate monitors, testing equipment, or the like) that a medical professional (otherwise described herein as medical personal, or the like) may use on the users within the first medical parking stations 1508. Alternatively, or additionally, the one or more second medical parking stations 1509 may operate as more private medical service stations 1510. As such, the one or more second medical parking stations may operate as more private medical bays, in which a user and/or the user's vehicle can be at least partially enclosed by a privacy partition (e.g., by doors, retractable screens, curtains, or the like) in order to provide more private medical services to the user, either within or outside of the user's vehicle.

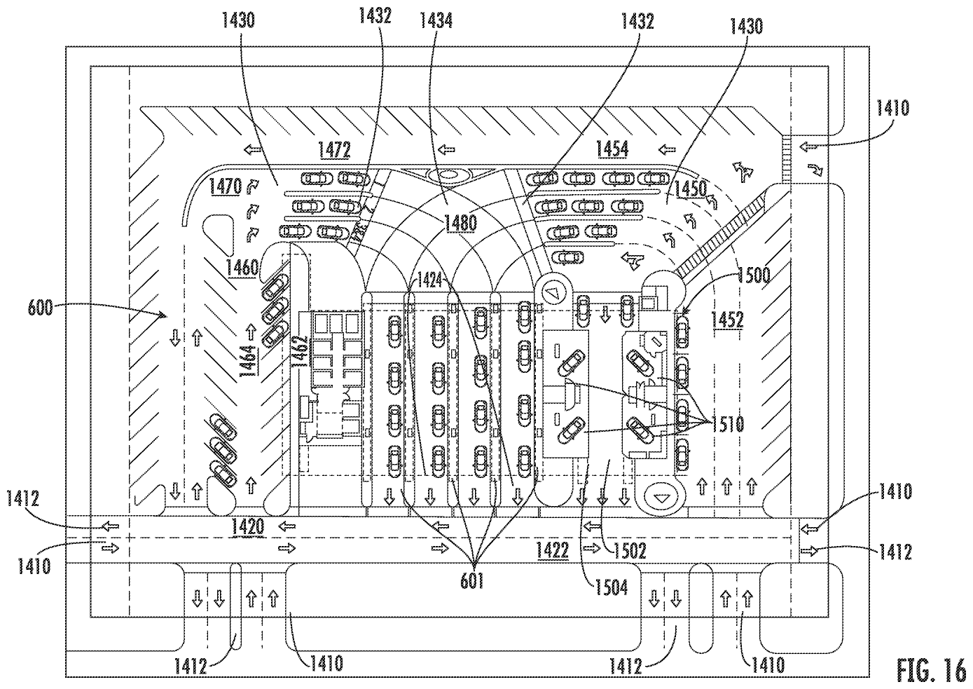

FIG. 16 illustrates another embodiment of the invention in which the medical services section 1500 may have one or more drive through lanes 1502 and/or one or more medical parking stations 1506. In the illustrated embodiment, the one or more medical parking stations 1506 may include only one or more private medical parking stations 1509 that operate as the one or more medical service stations 1510 (e.g., private medical bays), in which a user and/or the user's vehicle can be at least partially enclosed by a privacy partition (e.g., by doors, retractable screens, curtains, or the like) in order to provide medical services to the user, either within or outside of the user's vehicle. As previously described with respect to FIG. 15, the privacy bays may include the medical service devices (e.g., diagnostic equipment, thermometers, blood pressure monitors, heart rate monitors, testing equipment, or the like) that a medical professional may use on the users within the private medical parking stations 1509.

In alternate embodiments, the medical services section 1500 may operate as patient intake area in which users (e.g., patients, caregivers, or the like) provide receptionists and/or medical professional medical information in order to determine where the patients need to go, which will be described in further detail herein.

The medical services section 1500 may include ventilation equipment (e.g., blowers, suction, filters, climate control, or the like) that removes any exhaust fumes from the vehicles, air around the users and medical professional, or the like in order to create a more enjoyable environment around the users receiving medical services and/or the medical professionals providing the medical services.

The medical services section 1500 may further include barriers 1520 that prevent users from accessing the medical services stations 1510 until the users are approved (e.g., based on pre-approval to enter, the user has a scheduled appointment, or the like). Moreover, it should be understood that before, during, or after the user receives medical attention within the medical services sections 1500 an order may be placed for prescriptions and/or other products from other vendors, which may be delivered to the user as previously discussed herein (e.g., through the vertical transport systems, the robotic delivery systems 1110, the pick-up stations 622, or the like). As such, after receiving medical care, the user may drive to a pick-up station 622 to receive the order (e.g., if the order contains products other than prescriptions). However, it should be understood that in some embodiments pick-up stations 622 may be located within the medical services section 1500 and/or may be delivered directly to the medical services stations 1510 and provided to the user by medical personal during the user's visit (e.g., if the order only contains prescriptions, or related medical supplies, and/or over the counter medication from the pharmacy within the multi-vendor complex). In some embodiments of the invention, the order may be placed and directed to the medical services section 1500, such as directly to the medical service station 1510 as previously discussed herein (e.g., through the vertical transport systems, the robotic delivery systems 1110, the pick-up stations 622, or the like). However, it should be understood that the order may be directed to the medical services section 1500, such as directly to the medical service station 1510 through the use of pneumatic tubes, gravity delivery, drones, conveyors, or other like powered or non-powered delivery equipment and/or methods.

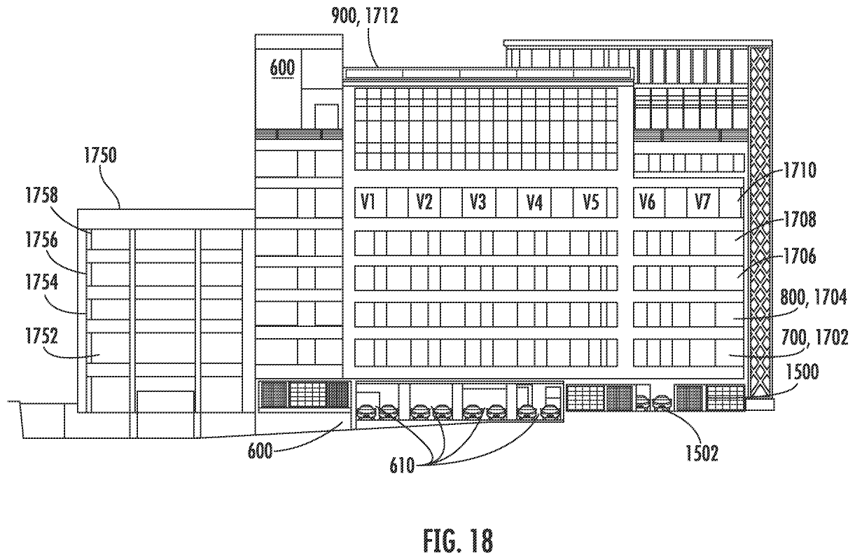

FIG. 18 illustrates other embodiments of the invention, in which the multi-vendor complex 600 includes the vendors previously described herein (e.g., V1-V7), that is, restaurants, fast food, fast casual, ghost kitchens, pharmacies, e-commerce institutions, clothing stores, hardware stores, home improvement store, grocery store, retail warehouses, convenience stores, electronic stores, lens/glasses stores, liquor stores, any type of retail store, and/or the like. However, FIG. 18 further illustrates that the multi-vendor complex 600 may have one or more additional floors 1700 that comprise offices and/or other facilities. In some embodiments, the offices may comprise organization offices and/or other facilities that may be rented to different organizations. In some embodiments, the offices and/or other facilities may include offices for medical personal and/or additional medical services (e.g., higher level surgeries, diagnostics that require longer wait times, treatments that require longer wait times, more invasive medical services, or the like). In some embodiments it should be understood that different floors of the multi-vendor complex 600 may offer different types of medical services. As such, in some embodiments a first medical floor 1702 may treat patients that have a first type of medical diagnosis, a second medical floor 1704 may treat patients that have a second type of medical diagnosis, a third medical floor 1706 may treat patients that have a third type of medical diagnosis, and a nth medical floor may treat patients that have an nth medical diagnosis. As such, in some embodiments, the first medical floor 1702 may include facilities and offices for medical professionals that treat patients that have communicable diseases, while the second medical floor 1704 may include facilities and offices for medical professionals that treat patients that have bone, muscle, tendon, skin, eyes, ears, nose, throat, vascular, or the like medical issues. Furthermore, the third medical floor 1706 may include facilities and offices for medical professional that treat cancer, autoimmune, or other like diseases. As such, it should be understood that the different floors 1700 of the multi-vendor complex 601 may be able to separate patients from other patients that could potentially harm each other. For example, patients with communicable diseases can be separated from patients that may have autoimmune diseases. It should be understood that the floors may be separated in any order and for any medical purpose.

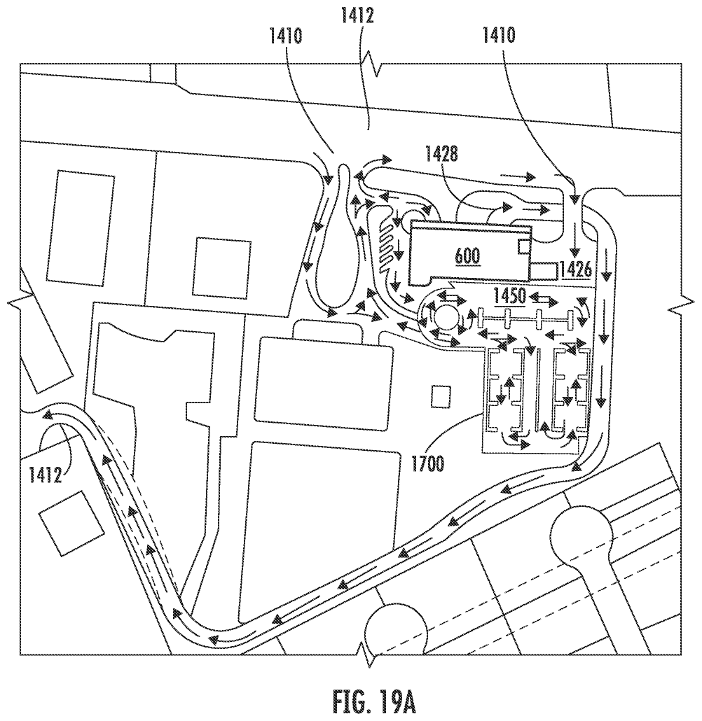

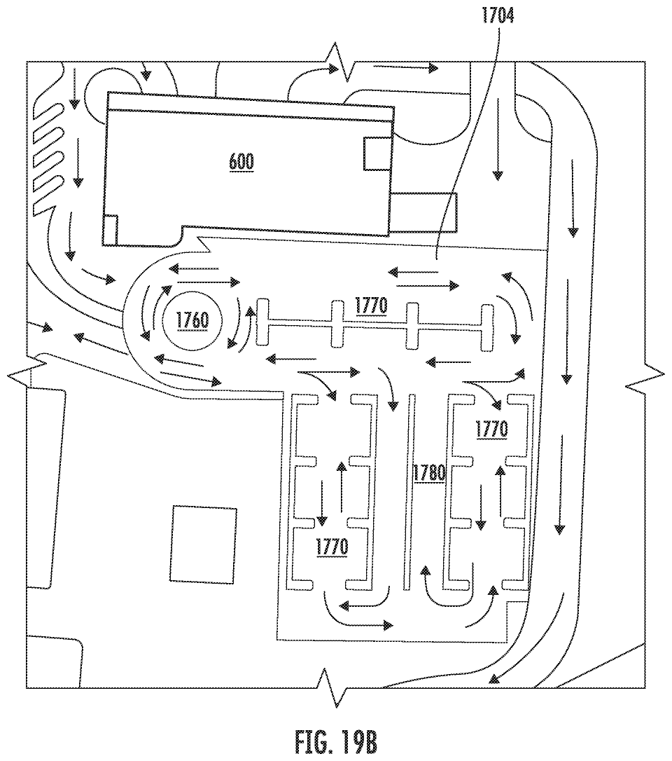

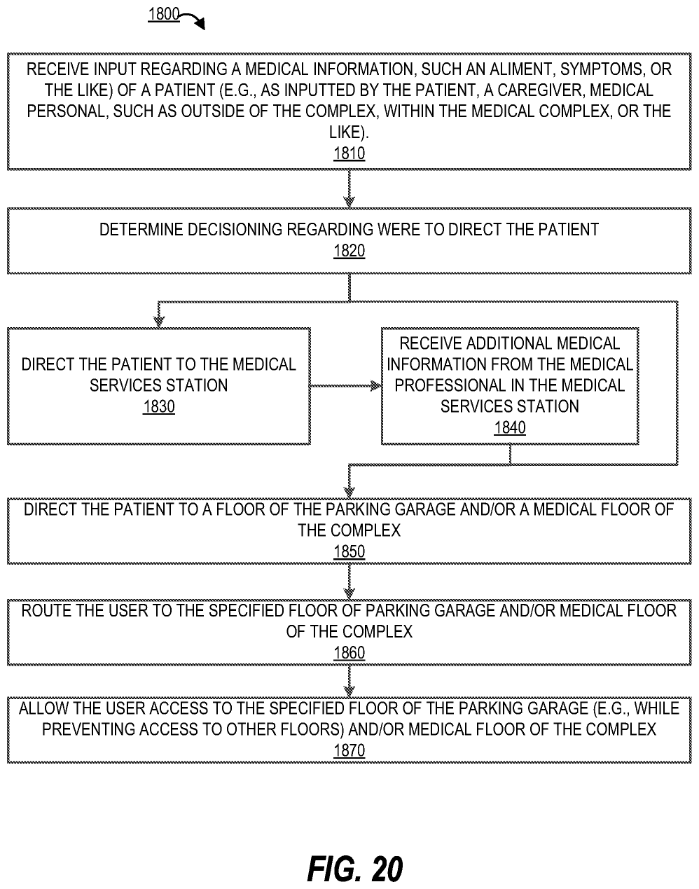

FIGS. 19A and 19B are similar to FIG. 17, but illustrates the traffic flow for the multi-vendor complex 600 and a floor of a parking garage 1750, in accordance with embodiments of the invention. As illustrated in FIGS. 19A and 19B, each of the floors 1751 of the parking garage 1750 may include a drop-off area 1760 which allows patients to be dropped off at the floor of the complex 600 for the particular medical issue of the patient. The drop-off area 1760 is illustrated as a circular drop-off area 1760, but it should be understood that it may be any type of drop-off area made of any shape. The parking garage 1750 may have any type of configuration, but in the illustrated embodiment, the parking garage 1750 may have flat floors 1770 (e.g., generally flat floors without large inclines, other than inclines to drain water, or the like) with inclined ramps 1780 located within the flat floors 1770 (as illustrated), located on the perimeter of the flat floors (not illustrated), helical ramps (not illustrated), or the like entry and exit ramps. It should be understood that in some embodiments, the patient and/or the vehicle in which the patient may be located may be directed to a particular floor of the plurality of floors 1751 of the parking garage 1750 depending on the medical condition of the patient. As such, depending on the medical information of the patient, such as the aliment, symptoms, previous diagnosis, diagnosis made in the medical services section 1500, or the like, the patient may be directed to a particular floor of the plurality of floors 1751 of the parking garage 1750, as will be described in further detail with respect to FIG. 20. In this way, for example, contagious patients may be separated based on medical information, such that some patients may be kept separate from other patients.

In some embodiments, the multi-vendor complex 600 may further comprise a plurality of elevators that may be utilized to move users between the floors of the complex 600. For example, instead of using a parking garage 1750, patients that may be walking to the complex 600 may utilize elevators to access different floors. In particular, with respect to the medical floors of the complex 600, one or more elevators may only be able to access one or more of the medical floors, while other separate elevators may only be able to access one or more other medical floors. For example, a first elevator may only access a first floor, a second elevator may only access a second floor, a third elevator may only access a third floor, and/or an nth elevator may only access an nth floor. As such, patients may only be able to access specific medical floors using specific elevators. In this way, like the parking garage 1750, contagious patients may be separated based on medical information, such that some patients may be kept separate from other patients, including when riding elevators.

In some embodiments, while the parking garage 1750 and/or the dedicated elevators may be used in the multi-vendor complex 600, it should be understood that the parking garage 1750 and/or the dedicated elevators may be used in a complex that is predominantly used for medical services (e.g., hospital, multi-medical vendor complex, or the like).

FIG. 20 provides a high level process flow for routing patients throughout the complex 600, and in particular, to different floors of the parking garage 1750 and/or medical floors of the multi-vendor complex 600, in accordance with embodiments of the invention. In some embodiments of the invention, as illustrated in block 1810 of FIG. 20, the medical application 383 (e.g., as previously described with respect to FIG. 3) may receive input regarding medical information. It should be understood, that the medical information may be inputted by any user, such as the patient, a caregiver for the patient (e.g., a family member, employee, or the like), and/or by a medical professional, such as a medical professional located off-site (e.g., at another medical facility, or the like) or on-site (e.g., should the patient call before coming on-site, or the like). The medical information may include symptoms the patent is having, aliments (e.g., prior, current, or the like), medical history, family medical history, insurance information, primary care information, or the like.

Block 1820 of FIG. 20 illustrates that the medical application 383 and/or system thereof, determines a decisioning action for the user (e.g., patient, caregiver aiding the patient, or the like) regarding what the user should do. For example, the medical application 383 (e.g., artificial intelligence, or the like) may automatically determine a decisioning action for the user. Additionally, or alternatively, a medical professional (e.g., a telehealth nurse, video nurse, or the like) may aid in making a decisioning action (e.g., receives the medical information through the medical application 383, over a call, or the like, and at least partially provides a recommended decisioning action). The decisioning action may be displayed to the user 110 on a user interface of the user computer system 400 and/or communicated to the user over a call (e.g., telephone, video call, or the like). In some embodiments, the medical application 383 and/or the medical professional may indicate to the user that no action is necessary. In some embodiments, if the medical information and/or the medical professional indicates that the medical services section 1500 and/or the higher level medical services provided within the medical floors of the complex 600, cannot treat the patient, the decision action may be to direct the user the emergency room, an open urgent care facility, the user's primary care physician, and may also allow the user to alert emergency room personal.

FIG. 20 further illustrates in block 1830, that the decisioning action may be to direct the patient to the medical services station 1500 through a user interface of the user computer system 400. In some embodiments, the decisioning action may include a time of an appointment for the user 110 to arrive at the medical services station 1500, may indicate that the patient may arrive immediately, or the like. The decisioning action may also include directions to arriving at the medical services stations 150 of the complex 600.

Alternatively, block 1850 further illustrates that the decisioning action may be to direct the user 110 directly to the a floor of the parking garage 1750 so that the patient may go directly to one of the medical floors of the complex 600 in order to receive medical attention that may not be available in the medical services section 1500 of the drive-through complex 600.

FIG. 20 further illustrates in block 1840, that when the patient is directed to the medical service section 1500, the medical application 383 may receive additional medical information from a medical professional within the medical services station 1500. For example, the medical professional may have access to the medical information of the patient (e.g., based on access to the patient's medical history, the medical information entered by a user into the medical application 383, and/or the like). The medical professional may analyze the patient within the medical services station 1500 and provide medical services as previously discussed herein (e.g., in the first medical parking stations 1508 and/or the one or more second medical parking stations 1509, or the like previously discussed herein). In some embodiments the medical professional, may add additional medical information to the patient's medical information within the medical application 383. In some embodiments, the medical services section 1500 may serve as the patient intake location at which patients are seen by a medical professional (e.g., nurse, or the like) before seeing another medical professional within one of the medical floors of the complex 600.

In some embodiments, after being seen by the medical professional within the medical services section 1500, the patient may be directed to leave (e.g., after the user has been treated), may be directed to the drive-through lanes 601 to receive a prescription or may receive the prescription in the drive-through lane, as previously described herein.

Alternatively, as previously discussed, when a patient is decisioned to go directly to a medical floor of the multi-vendor complex 600, or after seeing a medical professional within the medical services station 1500, block 1850 illustrates that the decisioning action may be to direct the user 110 to a medical floor of the complex 600 and/or a specific floor of the parking garage 1750. When the patient is to receive additional medical services (e.g., higher level medical services), the medical application 383 may receive an identifier that allows the patient to access a specific area of the parking garage 1750 and/or a specific medical floor 1700 of the complex 600. The identifier may be a code (e.g., barcode, QR code, character code, or the like), an electronic communication, or other identifier, that is the same or similar to the identifier that may be used to allow a user to access a locker at a pick-up station 622, as previously described herein. The identifier may be used by the patient to access different areas of the parking garage 1750 and/or a specific medical floor of the complex 600.

Block 1860 illustrates that the user may be directed to a specific floor of the parking garage 1750 that corresponds with the medical floor 1700 of the complex 600 at which the patient is to receive additional medical services (e.g., after being sent directly, after seeing a medical professional in the medical services section 1500, or the like). In some embodiments, the medical application 383 may route the user to the correct floor of the parking garage 1750 by displaying routing information on the interface of the user computer system 400. For example, the routing information may include the designated parking floor 1751 of the parking garage 1750 for the designated medical floor of the complex 600. The routing information may include a map of the parking garage 1750 and/or the medical floor of the complex 600.

FIG. 20 further illustrates in block 1870, that the medical application 383, through the use of the identifier, may allow the patient access to the specific floor 1751 of the parking garage 1750 and/or the specific medical floor 1700 of the complex 600. For example, the identifier may be used to gain access through a security feature (e.g., gate, locked door, locked barrier, or the like) within the parking garage 1750 and/or the floor 1700 of the complex 600. While the patient may be allowed to access a specific floor 1751 of the parking garage 1750 and/or a specific medical floor 1700 of the complex 600, the patient will not be able to access other floors of the parking garage 1750 and/or the other medical floors 1700 of the complex 600.

In some embodiments, instead of the medical application 383 allowing access to different locations of the parking garage 1750, the user may receive a device (e.g., electronic, RFID tag, NFC device, badge, or the like) that will only allow the patient to access the designed parking floor of the parking garage 1750 and/or the dedicated medical floor of the complex 600. As such, the user may only be allowed to access the designated parking floor and/or designated medical floor, while preventing the user from accessing non-designated parking floors of the parking garage 1750 and/or non-designated medical floors of the complex 600.

While the patient access to the medical floors of the complex 600 has been described herein as being controlled through the parking garage 1750, as previously described herein, there may be dedicated elevators that may only run to specific medical floors of the complex 600. As such, the users (e.g., patients, caregivers, or the like) use the medical application 383 (or another device) to access dedicated elevators for dedicated medical floors of the complex 600. As such, patients may be separated from each other, such as to prevent contagious patients from interacting with immune compromised patients.

Moreover, while the complex 600 described herein relates to a multi-vendor complex, the complex may be a dedicated medical complex that has the medical services section 1500, the parking garage 1750, and/or the medical floors 1700, with or without a pharmacy and/or the drive through lanes 601.

The present invention provides improvements for treating patients. For example, the present invention provides socially distanced healthcare for patients, including in particular, for patients that may be immune compromised and patents that may have communicable diseases. Additionally, the present invention provides high velocity throughput for patients seeking medical services, with little to no wait times. The complex also provides convenance through the use of access to other products (e.g., medical supplies, prescriptions, or the like) at the same location at which the patient receives medical treatment. Furthermore, the complex 600 allows medical services to be provided efficiently at lower costs, relieves strains on other medical facilities, and provides access to different levels of medical services at the same location through the use of improved technology, as described herein.

Alternatively, or additionally, to the medical services section 1500, the complex 600 may have a financial institution section at which users may perform banking interactions (e.g., withdraw funds, deposit funds, review accounts, open/close accounts, or the like). The financial institution section may include automated teller machines (ATMs), tellers, or the like at which users may undertake financial institution interactions. In some embodiments, the financial institution section may include interactive teller machines (ITMs) that provide video interactions with tellers along with the traditional functionality of ATMs. It should be understood that in some embodiments the pick-up stations 622, either in the traditional drive-through lanes 601 and/or in a dedicated financial institution section may combine the functionality of the pick-up stations 622 previously discussed herein along with the ability to enter into financial institution interactions. As such, in some embodiments, a user may enter into financial interactions before, during, or after receiving orders from a pick-up station 622. For example, a user may indicate that the user may want to enter into a financial transaction when the user is placing an order for one or more products with the one or more vendors of the complex (e.g., before, during, or after arriving at the complex). In some embodiments, the multi-vendor order fulfillment system 300 and/or other systems may factor in the financial institution interaction while determining when to deliver the products of the order to the pick-up station 622, when to direct the user to a pick-up station 622, determine a pick-up station 622 that includes ITM functionality to send the user to, or the like. In other embodiments, the user may enter into a financial institution interaction without any pre-ordering process, such as by using the ITM functionality of a pick-up station 622 during and/or after receiving and order from the pick-up station 622.

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, and the like), or an embodiment combining software and hardware aspects that may generally be referred to herein as a “system.” Furthermore, embodiments of the present invention may take the form of a computer program product on a computer-readable medium having computer-executable program code embodied in the medium.

Any suitable transitory or non-transitory computer readable medium may be utilized. The computer readable medium may be, for example but not limited to, an electronic, magnetic, optical, electromagnetic, infrared, or semiconductor system, apparatus, or device. More specific examples of the computer readable medium include, but are not limited to, the following: an electrical connection having one or more wires; a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device.

In the context of this document, a computer readable medium may be any medium that can contain, store, communicate, or transport the program for use by or in connection with the instruction execution system, apparatus, or device. The computer usable program code may be transmitted using any appropriate medium, including but not limited to the Internet, wireline, optical fiber cable, radio frequency (RF) signals, or other mediums.

Computer-executable program code for carrying out operations of embodiments of the present invention may be written in an object oriented, scripted or unscripted programming language such as Java, Perl, Smalltalk, C++, or the like. However, the computer program code for carrying out operations of embodiments of the present invention may also be written in conventional procedural programming languages, such as the “C” programming language or similar programming languages.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s).

The computer-executable program code may also be loaded onto a computer or other programmable data processing apparatus to cause a series of operational steps to be performed on the computer or other programmable apparatus to produce a computer-implemented process such that the code portions which execute on the computer or other programmable apparatus provide steps for implementing the functions/acts specified in the flowchart and/or block diagram block(s). Alternatively, computer program implemented steps or acts may be combined with operator or human implemented steps or acts in order to carry out an embodiment of the invention.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that steps of the processes described herein may be performed in orders different than those illustrated in the flowcharts. In other words, the processes represented by the blocks of a flowchart may, in some embodiments, be in performed in an order other that the order illustrated, may be combined or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A multi-vendor drive-through complex for multiple vendors providing goods to users, wherein the multi-vendor drive-through complex comprises:
one or more floors occupied by one or more vendors;
one or more drive-through lanes located below the one or more floors;
pick-up stations in the one or more drive-through lanes for storing orders, wherein the pick-up stations are automated pick-up stations, and wherein the orders comprise one or more goods provided by the one or more vendors; and
one or more vertical transport systems for delivering the orders to the one or more pick-up stations.

2. The multi-vendor drive-through complex of claim 1, wherein at least one floor of the one or more floors is occupied by two or more vendors.

3. The multi-vendor drive-through complex of claim 1, wherein the two or more vendors comprise food service providers operating separate kitchens.

4. The multi-vendor drive-through complex of claim 3, wherein at least a second floor of the one or more floors is occupied by a pharmacy.

5. The multi-vendor drive-through complex of claim 1, wherein the one or more floors may comprise one or more horizontal transport systems for transporting the one or more goods horizontally within the one or more floors.

6. The multi-vendor drive-through complex of claim 5, further comprising:
one or more consolidation stations on at least one floor of the one or more floors, wherein the one or more consolidation stations consolidate the one or more goods into the orders.

7. The multi-vendor drive-through complex of claim 1, wherein the orders comprise two or more sub-orders associated with two or more vendors.

8. The multi-vendor drive-through complex of claim 7, wherein the two or more sub-orders are fulfilled based on preparation times for the two or more sub-orders and order backlog of the two or more vendors.

9. The multi-vendor drive through complex of claim 8, wherein the two or more sub-orders are further fulfilled based on a location of the user.

10. The multi-vendor drive through complex of claim 1, wherein the users comprise end users or one or more third party users that pick-up the orders placed by the one or more end users.

11. The multi-vendor drive through complex of claim 1, wherein the pick-up stations have compartments that are adjustable to provide the orders to the users in user vehicles at different heights.

12. The multi-vendor drive through complex of claim 1, wherein at least one of the pick-up stations comprise at least a hot storage compartment and a cold storage compartment.

13. The multi-vendor drive through complex of claim 1, wherein the one or more drive-through lanes comprise two or more drive-through lanes and each of the two or more drive-through lanes have two or more pick-up stations.

14. A system for operating a multi-vendor drive-through complex, wherein the multi-vendor drive-through complex allows multiple vendors to provide goods to users, wherein the system comprises:
at least one network communication interface;
at least one non-transitory storage device; and
at least one processing device coupled to the at least one non-transitory storage device and the at least one network communication interface, wherein the at least one processing device is configured to:
receive orders from the users, wherein the orders comprise one or more goods;
consolidate and process the orders received from the users;
transmit the orders to vendors, wherein at least one order is transmitted to two or more vendors; and
determine that the orders are complete and operate one or more vertical transport systems to deliver the orders with the one or more goods to the pick-up stations, wherein the users pick-up the orders from the pick-up stations via one or more drive-through lanes.

15. A method for facilitating order placement, consolidation, and fulfillment of multi-vendor orders via a drive-through complex, the method comprising:
receiving an order from a user device of a user, wherein the order comprises at least two sub-orders from at least two vendors;
consolidating and processing the order received from the user;
identifying preparation times associated with the at least two sub-orders;
transmitting the preparation times to the two or more vendors through the vendor systems;
identifying a status of the at least two sub-orders;
determining an available drive-through lane with available pick-up station for the order with the at least two sub-orders; and
transmitting one or more notifications to the user or a third party user, wherein the one or more notifications are associated with at least one of the status of the order and the available pick-up station to pick-up the order.
